(19) United States
(12) Patent Application Publication
(10) Pub. No.: US 2026/0033745 A1
(43) Pub. Date: Feb. 5, 2026

(54) DETECTION OF GAIT ACTIVITY

(71) Applicant: HOFFMANN-LA ROCHE INC., Little Falls, NJ (US)

(72) Inventors: Lorenza ANGELINI, Basel (CH); Dimitar Yuriev STANEV, Basel (CH); Mattia ZANON, Basel (CH)

(21) Appl. No.: 19/298,828

(22) Filed: Aug. 13, 2025

Related U.S. Application Data
(63) Continuation of application No. PCT/EP2024/053661, filed on Feb. 14, 2024.

(30) Foreign Application Priority Data
Feb. 14, 2023 (EP) 23156442.8

(51) Int. Cl. A61B 5/11; A61B 5/00

(52) U.S. Cl. CPC A61B 5/112; A61B 5/4842; A61B 5/4848; A61B 5/6802; A61B 5/7264; A61B 5/7282

(57) ABSTRACT

The present invention relates to systems and methods for processing signals from wearable motion sensors associated with gait activity of a subject.

### FIELD OF INVENTION

The present invention relates to systems and methods for processing signals from wearable motion sensors associated with gait activity of a subject.

### BACKGROUND TO THE INVENTION

Neurological diseases such as multiple sclerosis (MS), Parkinson's disease (PD), Huntington's disease, often manifest themselves with symptoms like impaired posture and gait. These impairments are used as clinical indications of the disease progression. Precise assessments of gait abnormalities are particularly important to detect early-stage dysfunctions.

The use of technological devices such as wearable motion sensors at the ankles or in the lumbar region has greatly simplified gait detection and monitoring. The gyroscope and the accelerometer embedded in those sensors allow for measuring respectively the rotational changes and the linear changes relative to the frame of reference of the device. Methods exist that analyze gyroscope and accelerometer data to identify specific gait features of a subject, e.g. the characteristic motor patterns preceding the freezing of gait, i.e. the brief, episodic absence or marked reduction of forward progression typical of Parkinson's disease (Palmerini et al, 2017). However, while such methods can discriminate between rest and gait patterns, they have a number of limitations and fail to detect more granular gait features defining real-life gait activity.

However, these methods rely on the assumed location and/or orientation of the sensors on the subject to define the sensors' frame of reference. Any misplacement or misalignment of the sensors can result in inaccurate or wrong analysis results. Moreover, sensors placed at the same location on different subjects can have a different orientation depending on, for example, the subjects' body shape. Consequently, it is impossible to compare the gait features obtained with such methods among different subjects and/or sensors locations, in clinical settings as well as in unsupervised environments. Moreover, while state-of-the-art methods can discriminate between rest and general types of gait patterns such as normal and pathological gait, they fail to detect more granular gait features of clinical relevance.

Therefore there is a need for improved systems and methods for processing signals from wearable motion sensors associated with gait activity of a subject.

### STATEMENTS OF INVENTION

The present invention relates to systems and methods for processing signals from wearable motion sensors associated with gait activity of a subject. The methods may find particular use in the analysis of gait activity of a subject with a neurological dysfunction, such as Multiple Sclerosis (MS). These methods can be used, among other applications, to monitor disease progression, to assess the response of patients to a treatment in clinical studies, to compare the performance of a subject at a plurality of time points or the performance of different subjects in supervised or unsupervised environments.

The present inventors have identified that prior art methods for gait analysis are problematic because they rely on the assumed location and/or orientation of the sensors on the subject to define the sensors' frame of reference. Any misplacement or misalignment of the sensors can result in inaccurate or wrong analysis results. Moreover, sensors placed at the same location on different subjects can have a different orientation depending on, for example, the subjects' body shape. Consequently, it is impossible to compare the gait features obtained with such methods among different subjects and/or sensors locations, in clinical settings as well as in unsupervised environments.

The present inventors have identified that by leveraging specific operations on the signals received from wearable motion sensors, such as for example autocorrelations, sums and norms, it is possible to correctly decompose the signals in the triad of orthogonal components in the global walking directions, i.e. the vertical component along the direction of gravity, the antero-posterior component along the direction of walking, and the medio-lateral component. Processing the signals with the computer-implemented method of the present invention to identify said directional components thus allows to analyse gait activity of a subject independently of the position and/or orientation of the sensors on the subject.

Thus, according to a first aspect, the present invention provides a computer-implemented method of processing signals from one or more wearable motion sensors to analyse gait activity of a subject, the method comprising the steps of: receiving signals from the one or more wearable motion sensors located in any orientation on the subject, wherein the one or more wearable motion sensors comprise an accelerometer and a gyroscope; and processing the received signals, wherein processing comprises: dividing the signals from each sensor into a plurality of signal blocks comprising the signal of the respective sensor over a time interval; determining, for at least one of the plurality of signal blocks, directional components of the signal block, wherein determining directional components comprises: determining a vertical component of the signal block along the direction of gravity; estimating horizontal components of the signal block in the plane perpendicular to the direction of gravity, wherein the horizontal components consist of an antero-posterior component and a medio-lateral component; computing an autocorrelation of the vertical component of the signal block; computing an autocorrelation of each estimated horizontal component of the signal block; calculating the sum of the autocorrelations of each horizontal component and of the autocorrelation of the vertical component obtained in the previous steps; calculating the norm of the autocorrelations of each horizontal component and of the autocorrelation of the vertical component obtained in the previous steps; calculating the sum of the sum of autocorrelations and of the norm of autocorrelations obtained in the previous steps; computing an autocorrelation of the sum calculated in the previous step; estimating the location of the first peak of the autocorrelation obtained in the previous step; selecting, in each autocorrelation of the horizontal components, the peak located at the location of the first peak estimated in the previous step; validating the horizontal components of the signal blocks using one or more predetermined criteria that apply to the signs of the selected peaks of each autocorrelation of the horizontal components. The step of calculating the sum of the autocorrelations of each horizontal component and of the autocorrelation of the vertical component can be performed using Equation 1. The step of calculating the norm of the autocorrelations of each horizontal component and of the autocorrelation of the vertical component can be performed using Equation 2. The step of calculating the sum of the sum of autocorrelations and of the norm of autocorrelations can be performed using Equation 3. The step of validating the horizontal components of the signal block using one or more predetermined criteria that apply to the signs of the selected peaks of each autocorrelation of the horizontal components can comprise: determining, based on the results of evaluation of the one or more predetermined criteria, that the estimated horizontal components are correctly identified, and/or determining, based on the results of evaluation of the one or more predetermined criteria, that the estimated horizontal components are incorrectly identified. The said criteria are based on the biphasic nature of the antero-posterior component of a signal associated with gait activity and of the monophasic nature of the medio-lateral component of a signal associated with gait activity. In particular, the estimated horizontal components are correctly identified when: the selected peak of the antero-posterior component is positive and the selected peak of the medio-lateral component is negative; or the selected peak of the antero-posterior component and the selected peak of the medio-lateral component are negative and the selected peak of the antero-posterior component is the highest in absolute value. In particular, the estimated horizontal components are incorrectly identified when: the selected peak of the antero-posterior component is negative and the selected peak of the medio-lateral component is positive; or the selected peak of the antero-posterior component and the selected peak of the medio-lateral component are negative and the selected peak of the medio-lateral component is the highest in absolute value. In other words, the autocorrelation of the biphasic antero-posterior component should show a first positive peak while the autocorrelation of the monophasic medio-lateral component should show a first negative peak. If both first peaks are negative, the antero-posterior component should be selected as the component with the highest absolute value, given that the main direction of motion is the antero-posterior (i.e. forward/backward). The step of determining that the estimated horizontal components are incorrectly identified can comprise the step of identifying correctly the estimated horizontal components. In particular, the estimated antero-posterior component incorrectly identified corresponds to the correctly identified medio-lateral component and the estimated medio-lateral component incorrectly identified corresponds to the correctly identified antero-posterior component. In other words, once the horizontal components are incorrectly identified, they are swapped to be correctly identified.

The computed autocorrelations of the directional components contain information about the gait activity. In particular, the location of the first peak of the autocorrelations (positive for the vertical component and for the antero-posterior component, and negative for the medio-lateral component) quantifies the step duration. Similarly, the location of the second peak of the autocorrelations (positive for all three components) quantifies the stride duration. In the literature, a step frequency is estimated directly using the information of the peaks of such autocorrelations. However, the computed autocorrelations of the directional components can contain noise and/or spurious peaks, thus prejudicing the correct estimation of step duration and stride duration. Computing the sum and the norm of the autocorrelations of the directional components and autocorrelating their sum allows for noise removal and spurious peak rejection, thus improving the quantification of said gait features. Additionally, the location of the first peak of the autocorrelation of their sum can be used as a benchmark for validating the estimated directional components as hereinbefore described.

The method can have one or more of the following features.

The step of receiving signals from the one or more wearable motion sensors located in any orientation on the subject, wherein the one or more wearable motion sensors comprise an accelerometer and a gyroscope, can comprise receiving signals from the accelerometer and from the gyroscope comprised in the one or more wearable motion sensors. The step of receiving signals from the one or more wearable motion sensors located in any orientation on the subject, wherein the one or more wearable motion sensors comprise an accelerometer and a gyroscope, can comprise receiving signals from the accelerometer or from the gyroscope comprised in the one or more wearable motion sensors. The step of receiving signals from the one or more wearable motion sensors located in any orientation on the subject, wherein the one or more wearable motion sensors comprise an accelerometer and a gyroscope, can comprise receiving signals from the accelerometer in the one or more wearable motion sensors. The step of receiving signals from the one or more wearable motion sensors located in any orientation on the subject, wherein the one or more wearable motion sensors comprise an accelerometer and a gyroscope, can comprise receiving signals from the gyroscope in the one or more wearable motion sensors. Thus, any of the steps described below can be performed using signals received from the gyroscope, signals received from the accelerometer, or both.

The step of receiving signals from one or more wearable sensors located in any orientation on the subject can comprise receiving signals directly from one or more sensors, and/or receiving signals previously acquired by one or more sensors, from a user (e.g. through a user interface), from a computer, from a transmitting device, or from a data store. The step of receiving signals from one or more wearable sensors located in any orientation on the subject can comprise receiving signals previously obtained during one or more tests performed by the subject. The one or more tests can comprise active tests and/or passive tests. The one or more tests can have been performed in a controlled (e.g. clinic) or non-controlled (e.g. home) environment. An active test can be selected from: a test where walking is assessed at self-selected or imposed speed with or without periodic turns (e.g. every 10 meters), a test where static balance is assessed by asking the subject to remain in a static position under one of several conditions (e.g. eyes open, eyes closed) and in one or more positions (e.g. feet hip width apart, tandem stance), a test where turning and dynamic balance is assessed by asking the participant to perform predetermined movement patterns including walking and turning (e.g. U-turn every 4 steps, walking in FIG. 8, turn 360° counter-clockwise followed by 360° clockwise rotation). An active test can be selected from a 2MWT (2-Minute-Walking-Test), a U-Turn Test, and a SBT (Static Balance Test). A passive test can be referred to as passive monitoring. Passive monitoring refers to data collection taking place during daily life of the subject, excluding scripted tasks such as active tests. The subject can be a human subject. The subject can be an adult subject. The subject can be a paediatric subject. The subject can be an elderly subject, such as e.g. a subject of at least 60, 65, 70 or 75 years of age. The subject can be a healthy patient. The subject can be a subject that has been diagnosed as having a disease or disorder, being likely to have a disease or disorder, or being at risk of having a disease or disorder. In particular, the subject can be a subject that has been diagnosed with Alzheimer's disease, Parkinson's disease, MS, amyotrophic lateral sclerosis, Huntington's disease. In particular, the subject can be a subject likely to have Alzheimer's disease, Parkinson's disease, MS, amyotrophic lateral sclerosis, Huntington's disease. The received signals from one or more wearable sensors can be previously obtained while the subject is performing a single test or while the subject is performing a plurality of tests. The plurality of tests can be a plurality of tests of the same type or a plurality of tests comprising at least two different types of tests. Tests of the same type can be passive tests. Tests of the same type can be active tests. Tests of the same type can be 2MWT. The two different types of tests can be one active test and one passive test. The two different types of tests can be 2MWT and passive monitoring. The received signals can be previously obtained by tests performed by the subject repeatedly. The repeated tests can have been performed at regular intervals. The repeated tests can have been performed at random intervals. The received signals can be previously obtained in a supervised environment, for example a clinical setting. The received signals can be previously obtained in an unsupervised environment.

The step of dividing the signals from each sensor into a plurality of signal blocks comprising the signal of the respective sensor over a time interval can comprise dividing the signals from each sensor into a plurality of non-overlapping signal blocks and/or a plurality of signal blocks of a predetermined duration. The predetermined duration can be set as the mean step time of a reference population. The reference population can be a population of healthy individuals. From literature, the mean step time of a healthy population can be 0.5 seconds. The reference population can be a population diagnosed with a particular disease. The reference population can be a population monitored for a particular disease. The mean step time of a reference population diagnosed with a particular disease can be as high as 0.6/0.7 seconds, with a maximum value as high as 1.1 seconds, 1.5 seconds, 2 seconds.

The step of processing the received signal can further comprises: classifying one or more of a plurality of signal blocks between a plurality of categories comprising at least a rest category and a non-rest category, the non-rest category comprising at least a straight-gait category, using the magnitude and/or standard deviation of the respective signal block; selecting one or more of a plurality of signal blocks classified as at least one of the plurality of categories. The step of determining directional components can be performed on one or more of the selected signal blocks. A non-rest category can comprise a gait category and a turn category. A turn category can encompass blocks in which a turn of at least 90° is detected. A gait category can encompass blocks in which straight, semi-straight or curvilinear gait is detected. The selected signal blocks can be blocks classified in a non-rest category. The selected signal blocks can be blocks classified in a gait category. The step of determining, for at least one of the plurality of signal blocks, directional components of the signal block, can be performed on the selected signal blocks.

The present inventors have identified that by classifying parts of the signals, i.e. signal blocks, in categories based on the type of motion thereby encoded allows for a more accurate identification of clinically relevant gait features. For example, by selecting only signal blocks categorized as straight-gait blocks, it is possible to accurately infer, from the signal components along the global walking directions, gait features such as step duration, stride duration, step frequency. Further, by selecting only signal blocks categorized as straight-gait (or simply gait) blocks, it is possible to infer features associated with gait blocks (e.g. length of time, number of blocks, length of time in surrounding rest blocks, etc.) or the signal therein that are clinically relevant (i.e. usable as biomarkers). Similarly, by selecting only signal blocks categorized as turn blocks, it is possible to infer features associated with turn blocks (e.g. length of time, number of blocks, length of time in surrounding rest or straight-gait blocks, etc.) or the signal therein that are clinically relevant (i.e. usable as biomarkers). The clinical insights associated with gait blocks are expected to be different from those associated with turn blocks at least in some patients/conditions.

The present inventors have also identified that classifying parts of the signals, i.e. signal blocks, in categories based on the type of motion thereby encoded allows to validate the received signals. Thus, the method can comprise validating the received signal. Validating the received signals can comprise for example validating the output of a model applied to the raw signals to identify the type of motion encoded within the signals (e.g. a human activity recognition (HAR) model as described further below). For example, validating the received signals can comprise: performing a first classification of signal blocks by applying a human activity recognition model to the signal to classify the signal between a plurality of classes comprising one or more gait classes and one or more non-gait classes; performing a second classification of signal blocks by classifying one or more of a plurality of signal blocks between a plurality of categories comprising at least a rest category and a non-rest category as described herein; and comparing the first and second classification for one or more blocks. Blocks that are classified in predetermined categories that are not consistent with each other in the first and second classifications can considered as likely misclassified. A misclassified block can be excluded from further analysis, or one of the first or second classification may be ignored for this block as inaccurate. A block that is classified in the same category in the first and second classification, or in predetermined categories that are consistent with each other can be considered as validated.

Validating the received signals can comprise for example calculating the signal-to-noise ratio in the signals and discarding those part of the signals with low signal-to-noise ratio from further analysis. Validating the received signals can comprise for example verifying whether instructions were properly followed by the subject, using the categories assigned to one or more consecutive blocks under analysis. For example, if non-straight-gait blocks, and in particular turn blocks (i.e. blocks with turns above 90 degrees detected), are separated by gait blocks (i.e. blocks with no turn above 90 degrees and no rest detected, comprising straight-gait blocks, semi-straight-gait blocks or curvilinear blocks), the subject has followed correctly the test instructions and the received signals are valid. In particular, if the subject was requested to perform a 5 U-turn test (5UUT), in which the subject can walk at a self-selected comfortable pace and has to perform five consecutive 180 degrees turns (i.e. U-turns) at least 4 meters apart within 60 seconds, the timing and frequency of the blocks classified as U-turns separated by straight-gait blocks can be used to validate if the 5UUT instructions were properly followed. If, on the other hand, non-straight-gait blocks, and in particular U-turn blocks, are not separated by straight-gait blocks, the subject has not followed correctly the test instructions (e.g. has turned on the spot) and the received signals are not valid. Similar principles can also be applied in passive monitoring settings, for example where turn blocks can be validated as turn blocks only if they are adjacent to or within a predefined distance from gait blocks.

The present inventors have also identified that classifying parts of the signals, i.e. signal blocks, in categories based on the type of motion thereby encoded allows to infer information associated with the environment and/or context in which the subject was performing their gait activities, e.g. while performing a test in-clinic in a supervised environment or remotely in a self-administered mode and/or in an unsupervised environment, or else in passive monitoring conditions). In particular, said information associated with the environment and/or context can comprise features such as the number of turns, the number of walking bouts, the number and/or duration of rests, etc. Said information can allow to categorize or otherwise characterize the subject (e.g. identifying a level of severity of a disease or a symptom thereof) and/or to identify a subpopulation among the subjects tested. For example, subjects with a more advanced motor impairment rest more often and/or longer.

The rest category can comprise a short-rest category and a long-rest category, based on a predetermined rest duration threshold. In particular, signal blocks in the short-rest category can comprise signal blocks in the rest category with a duration below the predetermined rest duration threshold and signal blocks in the long-rest category can comprise signal blocks in the rest category with a duration above the predetermined rest duration threshold. A suitable rest duration threshold can be 2 seconds. A suitable rest duration threshold can be 2.5 seconds. A suitable rest duration threshold can be selected as a value between 1 and 3 seconds. The method can further comprise the step of merging signal blocks if spaced from each other below a predetermined time threshold. This step can serve to remove artifacts due to irregularity of the subject's movements throughout a test, in particular at the start and/or end of the test.

The non-rest category can comprise at least a straight-gait category. The non-rest category can comprise a semi-straight category. The non-rest category can comprise a curvilinear category. The straight-gait (or simply “gait”) category can comprise signal blocks with gyroscope signal above a predetermined minimum threshold. A suitable minimum threshold of the angular velocity measured by the gyroscope can be between 15 degrees per second and 30 degrees per second. The method can further comprise the step of classifying signal blocks in the non-rest category with a duration below a predetermined minimum duration as unknown blocks. Alternatively, the method can further comprise the step of classifying signal blocks in the non-rest category with a duration below a predetermined minimum duration as short gait blocks. A suitable minimum duration can be 2 seconds.

The method can further comprise classifying one or more of the plurality of signal blocks as boundary blocks. For example, signal blocks spaced from the start of the received signals or from the end of the received signals below a predetermined time threshold can be classified as boundary blocks. This step can serve to integrate the information about the context around a block.

The method can further comprise validating the signal using the classification of the plurality of signal blocks between the plurality of categories. Validating the signal using the classification of the plurality of signal blocks between the plurality of categories can comprise determining that the signal is invalid when the classifications assigned to a set of consecutive signal blocks does not satisfy one or more predetermined criteria. The one or more predetermined criteria can apply to the classification expected for a signal block that immediately follows or precedes a signal block of a particular type and/or to the minimum duration of a signal block or sets of signal blocks of a particular type. Validating the signal using the classification of the plurality of signal blocks between the plurality of categories can comprise comparing the classification assigned to a plurality of consecutive signal blocks with a predetermined set of consecutive classifications. The predetermined set of consecutive classifications can be associated with one or more tests associated with the received signal. A signal can be determined to be valid when the plurality of consecutive signal blocks match the predetermined set of consecutive classifications. The signal can be determined to be invalid when the plurality of consecutive signal blocks does not match the predetermined set of consecutive classifications. For example, a straight-gait block can be expected to be followed by a non-straight gait block or a rest block, and the total duration of a set of rest blocks or straight-gait blocks between two non-straight gait blocks can be expected to be at least a predetermined amount of time.

The method can further comprise validating one or more blocks identified as non-rest blocks using the output of a human activity recognition (HAR) model for the one or more blocks. The method can comprise applying a HAR model to a signal comprising a signal block identified as a non-rest block, and validating the block when the output of the HAR model satisfies one or more predetermined criteria or identifying the block as invalid when the output of the HAR model does not satisfy one or more predetermined criteria. A HAR model can be a machine learning model trained to classify wearable motion sensor signal between a plurality of classes comprising one or more gait classes and one or more non-gait classes. The HAR model can be a machine learning model trained in a supervised or unsupervised manner. The HAR model can be trained in a supervised manner, using wearable motion sensor data associated with labels corresponding to the plurality of classes. The one or more predetermined criteria can be criteria that apply to the proportion or percentage of time in the block that is classified in one or more predetermined classes (e.g. any gait class) by the human activity recognition model. For example, the HAR model can be configured to classify wearable motion sensor signal between a plurality of classes comprising a plurality of gait classes and a plurality of non-gait classes, and the one or more predetermined criteria can comprise a criterion that the percentage of time in the block classified in any of the plurality of gait classes by the HAR model is at or above a predetermined threshold.

According to a second aspect, the present invention provides a method according to the first aspect, further comprising, for at least one of the plurality of signal blocks, the steps of: calculating, using the determined directional components of the signal block, a step frequency; selecting one of the determined directional components of the signal block using the calculated step frequency and the computed autocorrelations of each directional component; obtaining a continuous wavelet transform of the selected directional component; calculating at least a first derivative of the continuous wavelet transform, and optionally a second derivative of the continuous wavelet transform; extracting, from the calculated at least first derivative and optionally second derivative of the continuous wavelet transform, one or more gait features. The one or more extracted gait features can comprise one or more of: step duration, stride duration, step frequency, stride frequency, stance-phase parameters (e.g. duration, average duration, frequency, average frequency, . . . ), swing-phase parameters (e.g. duration, average duration, frequency, average frequency, . . . ), percentage of stance-phase and swing-phase duration, step velocity, stride velocity, cadence, initial and terminal double support duration, single support duration, step length, stride length, and indirect gait features and unilateral versions of any of the above (e.g. gait-cycle events, heel-strike events, toe-off events, left/right heel-strike events, left/right stride duration, left/right step velocity). The method may further comprise determining the value of one or more statistics (e.g. predetermined percentile, mean, mode, median, predetermined quartile(s), standard deviation, etc.) derived from any one or more of the extracted gait features (such as e.g. 95^{th} percentile step velocity). Indirect gait features can comprise for example the subject's fatigue, stability, rhythm/variability, asymmetry, pace, forward balance, lateral balance, regularity, smoothness, intensity. Indirect gait features may be quantified at the level of one or more blocks. In an embodiment, the method according to the second aspect can be performed for at least one of the one or more classified and selected signal blocks. One or more of the above gait features can be used as biomarkers, for example when monitoring a subject (e.g. to determine symptoms and/or disease progression, or response to a treatment), selecting a subject for participating in a clinical trial or observational study, or providing a diagnosis or prognosis. For example, subjects with slower gait (e.g. more severe neurological disease) can spend more time in the stance phase (e.g. have longer stance-phase duration or average duration) than subjects with faster gait (e.g. less severe neurological disease). As another example, step velocity and/or stride velocity and statistics derived therefrom can be used as indicators of disease severity and response to treatment. Indeed, the 95^{th} percentile of stride velocity has been recently approved by the European Medicines Agency as a primary endpoint in studies in ambulatory Duchenne Muscular Dystrophy. Thus, metrics such as statistics of step velocity or stride velocity (whether unilateral or combining data over both sides) quantified using the methods described herein can be used as biomarkers in a variety of neurological diseases including in particular MS and PD. Thus, also described herein are methods of monitoring a subject who has been diagnosed as having or likely to have a neurological disease, the method comprising determining the value of one or more gait features for the subject as described herein. Also described herein is a method of diagnosing a subject as having a neurological disease or determining the severity of a neurological disease or motor symptom thereof in a subject, the method comprising determining the value of one or more gait features for the subject as described herein. Also described herein is a method of selecting a subject for participating in a clinical trial or observational study, the method comprising determining the value of one or more gait features for the subject as described herein, wherein the subject is selected for participating in the clinical trial or observational study when the determined values satisfy one or more predetermined criteria. Also described herein is a method of determining the effect of a treatment (such as e.g. administration of a therapeutic compound or composition), the method comprising determining the value of one or more gait features for a subject who has been administered the treatment as described herein. Any of the above methods can further comprise comparing the value of said gait features to respective comparative values, such as values obtained from the same subject at a preceding time point (e.g. before administration of a therapeutic), or values associated with a comparative or control group of subjects.

The step of calculating, using the determined directional components of the signal block, a step frequency can comprise the step of validating the calculated step frequency for one or more query signal blocks, wherein the step of validating the calculated step frequency can comprise the steps of: determining, for one or more neighbour signal blocks in a neighbourhood of the signal block, directional components of the neighbour signal blocks; calculating in the one or more neighbour signal blocks, using the determined directional components of the neighbour signal blocks, a neighbour step frequency; validating the calculated step frequency using the one or more calculated neighbour step frequencies, wherein the neighbourhood of a query signal block comprises bocks that are within a predetermined number of signal blocks away from the query block. The step of determining, for one or more neighbour signal blocks in a neighbourhood of the signal block, directional components of the neighbour signal blocks can be performed according to any aspect described herein. The neighbourhood of the query signal block can comprise a neighbourhood of 5, 10, 20, 30, or 50 signal blocks. The neighbourhood of the query signal block can comprise up to a predetermined number of signal blocks around the query signal block, with the query signal block as close as possible to the middle position of the series of blocks comprised in the neighbourhood. The neighbourhood of the query signal block can comprise one or two signal blocks immediately adjacent to the query signal block. For example, the neighbourhood of the query signal block can comprise the signal block immediately preceding the query block and/or the signal block immediately following the query block. Similarly, the neighbourhood of the query signal block can comprise up to a predetermined number of signal blocks immediately preceding the query block and/or a up to predetermined number of signal blocks immediately following the query block. The predetermined number can be e.g. 1,2, 5, 10, 20, 25 or 30 blocks. The neighbourhood can comprise “up to” predetermined numbers because the number of signal blocks available in a dataset (in total or preceding and/or following a query signal block) may be lower than the predetermined number. The neighbourhood of the query signal block can be limited to signal blocks that have been classified in a predetermined category. For example, the neighbourhood of the query signal block can be limited to signal blocks that have been classified as non-rest blocks. For example, the neighbourhood of the query signal block can comprise the non-rest signal block immediately preceding the query block and/or the non-rest signal block immediately following the query block. The query block can be a non-rest block. The step of calculating, in the one or more neighbour signal blocks, using the determined directional components of the neighbour signal blocks, a neighbour step frequency, can be performed according to any aspect described herein. The step of validating the calculated step frequency for a query block using the one or more calculated neighbour step frequencies can comprise comparing the calculated step frequency for the query block to the one or more neighbour step frequencies. The step of validating the calculated step frequency for a query block using the one or more calculated neighbour step frequencies can comprise comparing the calculated step frequency for the query block to the one or more neighbour step frequencies and determining (a) that the calculated step frequency for the query block is validated when the results of the comparison satisfy one or more predetermined criteria, or (b) that the calculated step frequency for the query block is not validated when the results of the comparison satisfy one or more predetermined criteria. The one or more criteria can comprise or consist of a criterion that applies to the size of the difference between the calculated step frequency for the query block and the one or more calculated neighbour step frequencies. The criterion can apply to the size of the difference between the calculated step frequency for the query block and an aggregate of the one or more calculated neighbour step frequencies. The criterion can apply to the magnitude of a probability comparing the calculated step frequency for the query block to a distribution of the one or more calculated neighbour step frequencies (e.g. probability that the calculated step frequency for the query block belongs to the same distribution as that associated with the one or more calculated neighbour step frequencies). For example, if the calculated step frequency is much larger or smaller than the neighbour step frequencies, the calculated step frequency can be corrected to be more similar to the neighbour step frequencies. In particular, the much larger or much smaller step frequency can be rejected as an outlier. A new step frequency can be calculated using an aggregate of the neighbour step frequencies. Suitable aggregates are mean, median, moving average, trimmed versions thereof. Thus, the step of validating the calculated step frequency for a query block using the one or more calculated neighbour step frequencies can comprise replacing the calculated step frequency for a query block that has been determined to not be validated with a new step frequency calculated using an aggregate of the neighbour step frequencies. Alternatively, a query block that has been determined as not validated may be excluded from further analysis. With a similar principle, neighbour step frequencies can be used to estimate the step frequency in signal blocks in which the step frequency is not calculable, for example in which the signal is missing and/or corrupted and/or mis-calibrated. Thus, the method can comprise identifying one or more query blocks for which a step frequency could not be calculated (e.g. a query block for which the step of calculating a step frequency resulted in a missing value), and calculating a step frequency for the query block using an aggregate of one or more neighbour step frequencies, wherein the one or more neighbour step frequencies are obtained by determining, for one or more neighbour signal blocks, directional components of the neighbour signal blocks; and calculating in the one or more neighbour signal blocks, using the determined directional components of the neighbour signal blocks, a neighbour step frequency, wherein neighbour signal blocks are signal blocks that are within a predetermined number of signal blocks away from the query block for which the calculated step frequency is being validated. Validation of step frequencies as described allows for a more robust estimation of the step frequency, particularly in the context of active tests. Validating the step frequency as described can be particularly advantageous in the context of active tests. In the context of passive monitoring, such validation can not be performed as the amount of available step data is typically higher (collected over longer periods of time) and the patient's movements are not as standardized. The step of calculating, using the determined directional components of the signal block, a step frequency can comprise the steps of: computing a first autocorrelation of the antero-posterior component of the signal block; computing a second autocorrelation of the antero-posterior component of the signal block, wherein the second autocorrelation consists of an autocorrelation of the first autocorrelation of the antero-posterior component obtained in the previous step; computing a first autocorrelation of the vertical component of the signal block; computing a second autocorrelation of the vertical component of the signal block, wherein the second autocorrelation consists of an autocorrelation of the first autocorrelation of the vertical component obtained in the previous step; computing a cross-correlation of the second autocorrelation of the antero-posterior component and of the second autocorrelation of the vertical component; estimating the distance between peaks of the computed cross-correlation; calculating, using the estimated distance, the step frequency. In an embodiment, the step of computing a cross-correlation of the second autocorrelation of the antero-posterior component and of the second autocorrelation of the vertical component comprises computing a cross-correlation of the sum of the second autocorrelation of the antero-posterior component and of the second autocorrelation of the vertical component.

In an embodiment, the step of calculating, in the one or more neighbour signal blocks, using the determined directional components of the neighbour signal blocks, a neighbour step frequency, can comprise the steps of: computing a first autocorrelation of the antero-posterior component of the neighbour signal block; computing a second autocorrelation of the antero-posterior component of the neighbour signal block, wherein the second autocorrelation consists of an autocorrelation of the first autocorrelation of the antero-posterior component obtained in the previous step; computing a first autocorrelation of the vertical component of the neighbour signal block; computing a second autocorrelation of the vertical component of the neighbour signal block, wherein the second autocorrelation consists of an autocorrelation of the first autocorrelation of the vertical component obtained in the previous step; computing a cross-correlation of the second autocorrelation of the antero-posterior component and of the second autocorrelation of the vertical component; estimating the distance between peaks of the computed cross-correlation; calculating, using the estimated distance, the neighbour step frequency. In a further embodiment, the step of computing a cross-correlation of the second autocorrelation of the antero-posterior component and of the second autocorrelation of the vertical component comprises computing a cross-correlation of the sum of the second autocorrelation of the antero-posterior component and of the second autocorrelation of the vertical component. The present inventors have identified that by leveraging a second autocorrelation of a first autocorrelation of the directional components, followed by the operation of cross-correlation, it is possible to estimate the stride duration from an estimate of the distance between every other peak of the cross-correlation. Such estimation of the stride duration is more precise than the one obtained by using first autocorrelations only as state-of-the-art methods do (Moe-Nilssen et al, 2004), since first autocorrelations can contain noise and/or spurious peaks. The step frequency calculated from the stride duration so obtained is thus also more precise than in state-of-the-art methods. Alternatively, the step frequency can be calculated from an estimate of the step duration, wherein the estimate of the step duration is obtained from an estimate of the distance between every peak of the cross-correlation. This is particularly the case when using the cross-correlation of the second autocorrelation of the antero-posterior component from the accelerometer signal and of the second autocorrelation of the vertical component from the accelerometer signal. Alternatively, the cross-correlation can be calculated as a cross-correlation of the sum of the second autocorrelation of the antero-posterior component from the gyroscope signal and of the second autocorrelation of the vertical component from the gyroscope signal. In such embodiments it is possible to estimate the stride duration from an estimate of the distance between every other peak of the cross-correlation. Indeed, when using the acceleration signal the cross correlation presents two peaks, one corresponding to step and one to stride. Such estimation of the stride duration is also more precise than the one obtained by using first autocorrelations only, and the inventors have additionally found it to be particularly robust and precise as the signal from the gyroscope is typically less noisy than the accelerometer signal for this purpose. The step frequency calculated from the stride duration so obtained is thus also more precise than in state-of-the-art methods.

Instead or in addition to this, a step frequency can be calculated using one or more other methods known in the art, such as any of the methods listed below.

In embodiments, the step of calculating, using the determined directional components of the signal block, a step frequency further comprises calculating one or more step frequencies using a plurality of different methods to obtain a plurality respective calculated step frequencies and selecting a calculated step frequency of the plurality of calculated step frequencies. For example, selecting a calculated step frequency can comprise selecting a calculated step frequency that is most common across the plurality of calculated step frequencies. When determining a calculated step frequency that is most common across the plurality of calculated step frequencies, one or more calculated step frequencies may be considered to be the same if they are within a predetermined tolerance of each other. The predetermined tolerance may be expressed in terms of values within a predetermined percentage of each other, values that are the same when rounded to a predetermined number of significant digits, or values that are within a predetermined range from each other. Suitable methods can be selected from sensor fusion methods, and in particular Kalman filter (Stratonovich, 1959), Dempster-Shafer algorithm (Dempster, 1967).

The continuous wavelet transform of the signals can allow to identify heel-strike events as the minima of the first derivative of the continuous wavelet transform. The continuous wavelet transform of the signal can allow to identify toe-off events as the maxima of the second derivative of the continuous wavelet transform. Alternatively, with an opposite wavelet sign, the heel-strike events can be identified as the maxima of the first derivative of the continuous wavelet transform and the toe-off events can be identified as the minima of the second derivative of the continuous wavelet transform. The present inventors have identified that the calculated step frequency can be functional to obtain the continuous wavelet transform, for example to obtain the parameterized continuous wavelet function that is applied to the signal to perform the continuous wavelet transform of the signal. In particular, the step frequency can be used directly to identify the wavelet axis and the wavelet scale. The wavelet axis can define the direction in which the continuous wavelet function is applied to the signal. The present inventors have identified that the wavelet axis can be identified as the direction of the directional component of the signal which corresponds to the highest power spectral density of the autocorrelation of the directional component of the signal in a neighbourhood of the calculated step frequency. The present inventors have also identified that the wavelet scale can be computed as a function of the calculated step frequency.

The method can have one or more of the following features.

The step of selecting one of the determined directional components of the signal block using the calculated step frequency and the computed autocorrelations of each directional component can comprise the steps of: calculating a power spectral density for each autocorrelation of each directional component; selecting an interval comprising the calculated step frequency; selecting the directional component with the highest power spectral density in the selected interval.

The step of obtaining a continuous wavelet transform of the selected directional component can comprise obtaining a parameterized continuous wavelet function, and obtaining the parameterized continuous wavelet function can comprise estimating at least a wavelet scale and/or a wavelet sign. The wavelet scale can be a function of the calculated step frequency. Estimating a wavelet sign can comprise the steps of: computing a norm of the accelerometer signal in the signal block; computing a correlation of the parameterized continuous wavelet function and of the calculated norm; determining, based on the sign of the computed cross-correlation, the wavelet sign. Estimating a wavelet sign can comprise the steps of: computing a norm of the accelerometer signal in the signal block; computing a correlation of the norm of the accelerator signal and the continuous wavelet function; computing a correlation between the medio-lateral component of the gyroscope signal and the continuous wavelet function; and computing the correlation between the norm of the accelerator signal and the medio-lateral component of the gyroscope signal.

The step of extracting, from the calculated at least first derivative and optionally second derivative of the continuous wavelet transform, one or more gait features can comprise identifying a plurality of heel strike events and determining, from said plurality of heel strike events, a gait feature selected from step velocity, stride velocity, and statistics derived therefrom. Determining a step or stride velocity can comprise determining one or more step lengths (e.g. a single step length when determining a step velocity, and the length of two consecutive steps when determining a stride velocity) and a step or stride duration. Determining a step or stride duration can comprise determining the amount of time between two consecutive identified heel strike events (step duration) or between two identified heel strike events on the same side (stride duration). Determining a step length can comprise integrating the acceleration signal from a wearable motion sensor twice to determine a path (i.e. position as a function of time) of the sensor, determining a displacement of the sensor during the step from said determined path, and calculating a step length using said displacement. The calculation can use a known or estimated vertical distance between the sensor and the floor (height of the sensor) when the subject is standing at rest. The sensor can be located in the vicinity of the centre of mass (e.g. anywhere around the waist or hips of the subject). In such embodiments, the path can be referred to as the path of the subject's centre of mass (COM), and the displacement can be referred to as the displacement of the subject's COM. Use of a sensor in the vicinity of the subject's COM enables the distance between the sensor and the floor to be estimated as the known or estimated length of the leg of the subject. In embodiments where the length of the leg of the subject is not known and the sensor is located in the vicinity of the centre of mass, the height of the sensor can be estimated based on the height of the subject and an anthropometric relationship (i.e. a predetermined relationship between subject height and leg length). In embodiments, a height of the sensor (e.g. leg length) can be assumed as predetermined value independent of the subject. This can result in less accurate determination of step lengths values but the step lengths values can still be used to assess changes in step lengths (e.g. longitudinal changes in a subjects, effects of fatigue or treatment, etc). Calculating a step length can use Equation (6) below, where dCOM is the displacement of the centre of mass during the step and l_{leg} is parameter corresponding to the sensor height (vertical distance between the sensor and the floor when the subject is standing at rest, also referred to as the length of the leg of the subject in embodiments where the sensor is located in the vicinity of the subject's centre of mass). Integrating the acceleration signal can comprise integrating the vertical component of the acceleration signal or the magnitude of the acceleration signal. The latter can result in more robust estimates of step length. Integrating the acceleration signal can comprise re-initialising a numerical integration of the acceleration signal for each consecutive step of a plurality of steps using as initial conditions the velocity and/or position estimate obtained by integrating the signal for the preceding step.

The step of extracting, from the calculated at least first derivative and optionally second derivative of the continuous wavelet transform, one or more gait features can comprise identifying a plurality of heel strike events and determining whether a heel strike event is a left heel strike event or a right heel strike event based on the sign of a signal corresponding to the angular velocity around the vertical axis derived from a received gyroscope signal. Such embodiments can comprise obtaining, from the gyroscope signal, a signal corresponding to the vertical angular velocity of the sensor and determining whether a previously identified heel strike event is a left heel strike event or a right heel strike event based on the sign of said signal at the time of the identified heel strike event. In embodiments, a heel strike event can be identified as a left heel strike event when the signal is positive at the time of the heel strike event, and a heel strike event can be identified as a right heel strike event when the signal is negative at the time of the heel strike event.

A step velocity can be calculated as the ratio of the step length to the step duration. A stride velocity can be calculated as the ratio of the stride length to the stride duration. A stride length can be calculated as the sum of a left/right step length and a right/left step length. Determining a step velocity can comprise determining a step velocity for one or more left steps, wherein a left step is a step between a right heel strike and a left heel strike. Determining a step velocity can comprise determining a step velocity for one or more right steps, wherein a right step is a step between a left heel strike and a right heel strike.

The step of extracting, from the calculated at least first derivative and optionally second derivative of the continuous wavelet transform, one or more gait features can comprise the steps of extracting gait-cycle events and validating the extracted gait-cycle events. A gait-cycle event can be selected from: a left heel strike, a left toe off, a right toe off and a right heel strike. The step of validating the extracted gait-cycle events can comprise determining whether the extracted gait-cycle events satisfy one or more predetermined criteria derived from the expected order of gait-cycle events in a gait cycle. For example, the step of validating the extracted gait-cycle events can comprise determining whether the extracted gait-cycle events comprise two or more consecutive gait-cycle events of the same type (e.g. two consecutive left toe-off events, indicating that one of the two is likely not a valid gait-cycle event, i.e. false positive gait cycle event). Each instance of such two or more consecutive gait-cycle events of the same type can be identified as including at least one extracted gait-cycle event that is not valid. Instead or in addition to this, the step of validating the extracted gait-cycle events can comprise determining whether the extracted gait-cycle events comprises a set of gait-cycle events that does not follow a predetermined order of types of gait-cycle events. For example, sets of gait-cycle events comprising a right heel strike followed by a left heel strike may be identified as an invalid gait cycle, such as a gait-cycle that is missing a gait cycle event (false negative gait cycle event). In embodiments, the predetermined order can be: right heel strike, left toe-off, left heel strike, right toe off, and any order that is obtained by cycling through these in the provided order (e.g. left toe-off, left heel strike, right toe off, right heel strike, etc.). The step of validating the extracted gait-cycle events can comprise the steps of: identifying false positive and/or false negative gait-cycle events; removing false positive and/or false negative gait-cycle events; and/or validating the extracted gait-cycle events using gait cycle events in one or more neighbour gait-cycles. Identifying false positives gait-cycle events can comprise identifying two or more consecutive gait-cycle events of the same type. Identifying false negative gait-cycle events can comprise identifying a set of gait-cycle events comprising one or more missing gait-cycle events between gait cycle events that satisfy a predetermined order of types of gait-cycle events. Removing false positive and/or false negative gait-cycle events can comprise removing false positive and/or false negative gait-cycle events using neighbour gait cycles. Removing false-positive gait-cycle events using neighbour gait cycles can comprise determining which of a set of consecutive gait-cycle events identified as including a false positive gait-cycle event is most likely to be a false positive using the locations of corresponding gait-cycle events in one or more neighbour cycles and removing said gait-cycle event. Removing false-negative gait-cycle events using neighbour gait-cycle events can comprise determining a likely location of a gait-cycle event identified as a false negative using the locations of corresponding gait-cycle events in one or more neighbour gait cycles and imputing the false negative gait-cycle event at the determined likely location. Removing false positive gait-cycle events can further comprise selecting the gait-cycle events based on one or more of the following parameters: loading response duration, stance duration, stride duration. In particular, the gait-cycle events can be selected based on a loading response duration smaller than a maximum loading response duration threshold, and/or on a stance duration smaller than a maximum stance duration threshold, and/or on a stride duration smaller than a maximum stride duration threshold. The maximum loading response duration threshold and/or the maximum stance duration threshold and/or the maximum stride duration threshold can be functions of the estimated step frequency. Neighbour gait-cycle events can comprise gait-cycle events temporally preceding each extracted gait-cycle event. Neighbour gait cycles can comprise gait cycles temporally preceding the gait cycle of an extracted gait-cycle event. Neighbour gait-cycle events can comprise gait-cycle events temporally following each extracted gait-cycle event. Neighbour gait cycles can comprise gait cycles temporally following the gait cycle of an extracted gait-cycle event. Neighbour gait-cycle events can be 1, 2, 5, or 10 gait-cycle events temporally preceding or following each extracted gait-cycle event. Neighbour gait cycles can be 1, 2, 5, or 10 gait cycles temporally preceding or following the gait cycle including an extracted gait cycle event. For example, if a gait-cycle event is missing in an extracted gait cycle (e.g. a gait cycle comprises gait-cycle events that do not follow a predetermined order), temporally preceding (or following) gait cycles can be used to determine the most probable position of the missing event. As another example, if an extra gait-cycle event is extracted in a gait cycle (e.g. a gait cycle comprises two consecutive gait-cycle events of the same type), temporally preceding (or following) gait cycles can be used to determine the most probable position of the correct gait-cycle event (e.g. the one of the two consecutive gait-cycle events of the same type that is more likely to be correct). This step allows for a more robust extraction of gait features, and in particular gait-cycle events. A gait cycle can be determined as a set of gait cycle events together forming consecutive left and right steps.

According to a third aspect, there is provided a method of diagnosing or monitoring a neurological dysfunction associated with gait activity in a subject, the method comprising analyzing the gait activity of the subject using the method of any embodiment of the preceding aspects. The neurological dysfunction can be selected from: multiple sclerosis (MS), Parkinson's disease (PD), Huntington's disease. The neurological dysfunction can be MS. The neurological dysfunction can be PD. Analyzing the gait activity can comprise determining one or more of: step duration, stride duration, step frequency, stride frequency, stance-phase parameters (e.g. duration, average duration, frequency, average frequency, . . . ), swing-phase parameters (e.g. duration, average duration, frequency, average frequency, . . . ), step velocity, stride velocity, indirect gait features unilateral versions of any of the above (e.g. gait-cycle events, heel-strike events, toe-off events, left/right heel-strike events, left/right stride duration, left/right step velocity), and statistics (e.g. predetermined percentile, mean, mode, median, predetermined quartile(s), standard deviation, etc.) derived from any of the above (such as e.g. 95^{th} percentile step velocity). Analyzing the gait activity can comprise determining the amount or proportion of time corresponding to signal blocks classified in one or more predetermined categories. For example, subjects with more severe motor disorder are expected to spend more time in rest blocks than subjects with less severe motor disorder. Indirect gait features can comprise for example the subject's cadence, fatigue, stability, rhythm/variability, asymmetry, pace, forward balance, lateral balance. It is proven in the literature that said gait features can provide diagnostic information for neurological dysfunctions (Angelini et al, 2021). Analyzing the gait activity can comprise determining one or more gait features (such as for example pace) and comparing the one or more gait features to one or more reference values. The one or more reference values can comprise an expected value of a gait feature associated with a healthy population (e.g. mean value previously determined for a healthy population). The one or more reference values can comprise an expected value of a gait feature associated with a diseased population (e.g. mean value previously determined for a diseased population). The one or more reference values can comprise a value of a gait feature previously obtained for the same subject.

According to a fourth aspect, there is provided a method of treating a subject for a neurological dysfunction, the method comprising: determining whether the subject has the neurological dysfunction using the method of any embodiment of the third aspect; and administering a therapeutically effective amount of a therapy for the treatment of the neurological dysfunction to the subject who has been determined as having the neurological dysfunction.

Also described herein is a computer-implemented method, comprising: receiving, by one or more processor, a signal from one or more sensors located on a subject, the signal representative of a gait activity of the subject; identifying, by the one or more processors, one or more directional components of the signal, the one or more directional components being independent of an orientation of the one or more sensors on the subject; extracting, by the one or more processors and based on at least on the one or more directional components of the signal, one or more gait features from the signal; and determining, by the one or more processors and based at least on the one or more gait features, at least one of a diagnosis, a progression, and a treatment response for a neurological dysfunction. The method can have any of the features described in relation to any embodiment of any preceding aspect.

For example, the extracting the one or more gait features can include calculating, based at least on the one or more directional components of the signal, a step frequency, determining, based at least on the step frequency, a wavelet transform of the one or more directional components; and determining, based at least on the wavelet transform, the one or more gait features. The determining the step frequency can include determining a first vertical autocorrelation of a vertical component of the signal and a second vertical autocorrelation of the first vertical autocorrelation, determining a first antero-posterior autocorrelation of an antero-posterior component of the signal and a second antero-posterior autocorrelation of the first antero-posterior autocorrelation, determining a cross-correlation of the second vertical autocorrelation and the second antero-posterior autocorrelation, and determining, based at least on a distance between two or more peaks in the cross-correlation, the step frequency. The signal can be an accelerometer signal. The signal can be a gyroscope signal. In such embodiments, determining the step frequency can include determining a first vertical autocorrelation of a vertical component of the signal and a second vertical autocorrelation of the first vertical autocorrelation, determining a first antero-posterior autocorrelation of an antero-posterior component of the signal and a second antero-posterior autocorrelation of the first antero-posterior autocorrelation, determining a cross-correlation of the sum of the second vertical autocorrelation and the second antero-posterior autocorrelation, and determining, based at least on a distance between two or more peaks in the cross-correlation, the step frequency.

The determining the wavelet transform can include convoluting, at successive timepoints, a wavelet across one directional component of the signal having a higher power in a frequency interval of the autocorrelation of the directional component of the signal than other directional components of the signal. The determining the wavelet transform can further include calculating, for each directional component of the signal, a power density spectrum of the autocorrelation of the signal, selecting, as the frequency interval, a range of frequencies within a threshold of the step frequency, and identifying, based at least on the power density spectrum of each directional component of the signal, the one directional component of the signal having the higher power in the frequency interval of the step frequency than the other directional components of the signal.

The method can further comprise determining the wavelet sign as described herein, for example in relation to the preceding embodiments. The method can further comprise: determining, based at least on the wavelet transform, one or more of a toe-off event and a heel-strike event; and determining, based at least on the one or more of the toe-off event and the heel-strike event, the one or more gait features. The one or more gait features can be determined based at least on a timing and/or a location of the one or more of the toe-off event and the heel-strike event. The heel-strike event can be determined as a minimum (or maximum, depending on the sign of the wavelet transform) of a first derivative of the wavelet transform. The toe-off event can be determined as a maximum (or minimum, depending on the sign of the wavelet transform) of a second derivative of the wavelet transform. The one or more gait features can include at least one of cadence, fatigue, stability, rhythm, variability, asymmetry, pace, forward balance, and lateral balance. The method can further comprise: dividing the signal into one or more signal blocks; and identifying, for each signal block, a vertical component, an antero-posterior component, and a medio-lateral component of the signal in the signal block. The method can further comprise validating the medio-lateral component and the antero-posterior component of identified for each signal block; and in response to a failure to validate the medio-lateral component and the antero-posterior component identified for a signal block, swapping the medio-lateral component and the antero-posterior component of the signal block. Validating the medio-lateral component and the antero-posterior component identified for each signal block can include: determining a medio-lateral autocorrelation of the medio-lateral component and an antero-posterior autocorrelation of the antero-posterior component, and validating, based at least on the medio-lateral autocorrelation and the antero-posterior autocorrelation, the medio-lateral component and the antero-posterior component identified for each signal block. Said validating the medio-lateral component and the antero-posterior component can comprise: determining a sum of the medio-lateral autocorrelation and antero-posterior autocorrelation, determining a norm of the medio-lateral autocorrelation and antero-posterior autocorrelation, determining a cross correlation of said sum and norm, and validating the medio-lateral component and the antero-posterior component based on said cross-correlation. Validating the medio-lateral component and the antero-posterior component identified for each signal block can further include: validating the medio-lateral component and the antero-posterior component of a signal block as correctly identified based at least on (i) the antero-posterior autocorrelation showing a first positive peak while the medio-lateral autocorrelation is showing a first negative peak, or (ii) a first peak of the antero-posterior autocorrelation having a highest absolute value where the first peak the antero-posterior autocorrelation and that of the medio-lateral autocorrelation are negative.

The method can further comprise classifying each signal block as a rest block or a non-rest block; and identifying the one or more directional components of the signal in one or more non-rest blocks and not one or more rest blocks. The classifying each signal block as a rest block or a non-rest block can include classifying, based at least on a magnitude of an accelerometer signal and/or a gyroscope signal in a signal block, the signal block as a rest block or a non-rest block.

The method can further comprise classifying, based at least on a presence of motion of a threshold magnitude in a single direction of movement or multiple directions of movement, each non-rest block as a straight-gait block or a non-straight gait block; and identifying the one or more directional components of the signal in one or more straight gait blocks and not one or more non-straight gait blocks.

The method can further comprise applying one or more of an orientation filter, a statistical analysis, and a sensor fusion to identify the one or more directional components of the signal.

According to a further aspect, there is provided a system of a processor, and a computer readable medium comprising instructions that, when executed by the processor, cause the processor to perform the computer-implemented steps of the method of any preceding aspect. The system can further comprise means for acquiring signals associated with a gait activity of a subject, for example one or more wearable sensors.

For example, also provided according to the present aspect is a system comprising at least one data processor; and at least one memory storing instructions, which when executed by the at least one data processor, result in operations comprising: receiving, from one or more sensors located on a subject, a signal representative of a gait activity of the subject; identifying one or more directional components of the signal, the one or more directional components being independent of an orientation of the one or more sensors on the subject; extracting, based at least on the one or more directional components of the signal, one or more gait features from the signal; and determining, based at least on the one or more gait features, at least one of a diagnosis, a progression, and a treatment response for a neurological dysfunction. The instructions can cause the processor to execute the method of any embodiment of any preceding aspect.

According to a further aspect, there is provided a non-transitory computer readable medium or media comprising instructions that, when executed by at least one processor, cause the at least one processor to perform the method of any embodiment of any aspect described herein.

Thus, also described according to the present aspect is a non-transitory computer readable medium storing instructions, which when executed by at least one data processor, result in operations comprising: receiving, from one or more sensors located on a subject, a signal representative of a gait activity of the subject; identifying one or more directional components of the signal, the one or more directional components being independent of an orientation of the one or more sensors on the subject; extracting, based at least on the one or more directional components of the signal, one or more gait features from the signal; and determining, based at least on the one or more gait features, at least one of a diagnosis, a progression, and a treatment response for a neurological dysfunction.

According to a further aspect, there is provided a computer program comprising code which, when the code is executed on a computer, causes the computer to perform the method of any embodiment of any aspect described herein.

### BRIEF DESCRIPTION OF THE FIGURES

FIG. 1 illustrates an embodiment of a system that can be used to implement one or more aspects described herein.

FIG. 2 is a flow diagram showing, in schematic form, a method of processing signals from one or more wearable motion sensors to analyse gait activity of a subject, according to the invention.

FIG. 3 is a flow diagram showing, in schematic form, a method of extracting one or more gait features, according to the invention.

FIG. 4 is a flow diagram showing, in schematic form, a method of calculating the step frequency of a subject, according to the invention.

FIG. 5 is a flow diagram showing, in schematic form, a method of selecting the wavelet axis of a continuous wavelet function, according to the invention.

FIG. 6 is a flow diagram showing, in schematic form, a method of estimating the wavelet sign of a continuous wavelet function, according to the invention.

FIG. 7 is a summary figure showing the methods of the previous figures relative to each other, according to the invention.

FIG. 8a shows the autocorrelations of the antero-posterior, medio-lateral, and vertical components of signals in a straight-gait block obtained from a wearable motion sensor on a subject, according to the invention.

FIG. 8b shows the sum, the norm, and the autocorrelation of the summed sum and norm of the antero-posterior, medio-lateral, and vertical components of signals in a straight-gait block obtained from a wearable motion sensor on a subject, according to the invention.

FIG. 9a shows the second autocorrelations of the antero-posterior and vertical components of signals in a straight-gait block obtained from a wearable motion sensor on a subject, according to the invention.

FIG. 9b shows the cross-correlations of the second autocorrelations of the antero-posterior and vertical components of signals in a straight-gait block obtained from a wearable motion sensor on a subject, according to the invention.

FIG. 10 illustrates a human gait cycle.

FIG. 11 illustrates the global walking directions.

FIG. 12A shows a diagram illustrating the concepts of steps and centre of mass path and displacement.

FIG. 12B shows a flow diagram illustrating, in schematic form, a method of calculating the step velocity of a subject, according to the disclosure.

FIG. 13 shows example results of an embodiment of a method of processing signals from one or more wearable motion sensor to analyse gait activity of a subject, illustrating the effect of the choice of thresholds used to classify blocks as rest vs non-rest blocks.

FIG. 14A shows two examples of incorrectly detected steps while walking, that are discarded once post-processing step is applied using a human activity recognition (HAR) model, according to the disclosure.

FIG. 14B shows two examples of correctly detected steps while walking, that are validated once the post-step using a HAR model is applied, according to the disclosure.

### DETAILED DESCRIPTION

In describing the present invention, the following terms will be employed, and are intended to be defined as indicated below.

A neurological dysfunction as used herein refers to a neurological condition or disorder that affects brain functions, in particular motor functions, including neurodevelopmental disorders, psychiatric disorders, neurodegenerative disorders. Examples of neurodegenerative disorders are Alzheimer's disease, Parkinson's disease, MS, amyotrophic lateral sclerosis, Huntington's disease.

The systems and method described herein can be implemented in a computer system, in addition to the structural components and user interactions described. As used herein, the term “computer system” includes the hardware, software and data storage devices for embodying a system and carrying out a method according to the described embodiments. For example, a computer system can comprise one or more central processing units (CPU) and/or graphics processing units (GPU), input means, output means and data storage, which can be embodied as one or more connected computing devices. Preferably the computer system has a display or comprises a computing device that has a display to provide a visual output display. The data storage can comprise RAM, disk drives, solid-state disks or other computer readable media. The computer system can comprise a plurality of computing devices connected by a network and able to communicate with each other over that network. It is explicitly envisaged that computer system can consist of or comprise a cloud computer. The wearable motion sensors or devices described herein can comprise sensors integrated into wearable objects, such as for example smartwartches, and/or into objects that can be carried by a subject, such as for example smartphones, tablets, laptops, Inertial Measurement Units (IMU), and/or directly within the body, such as for example subcutaneous chips. The wearable motion sensors described herein comprise an accelerometer and a gyroscope. The wearable motion sensors described herein are configured to transmit data to a computer system.

As used herein “data” and “signals” are used interchangeably unless otherwise specified.

The methods described herein are computer implemented unless context indicates otherwise. Indeed, the features of the data associated with gait activity are such that the methods described herein are far beyond the capability of the human brain and can not be performed as a mental act. The methods described herein can be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media, magnetic tape; optical storage media such as optical discs or CD-ROMs; electrical storage media such as memory, including RAM, ROM and flash memory; hybrids and combinations of the above such as magnetic/optical storage media.

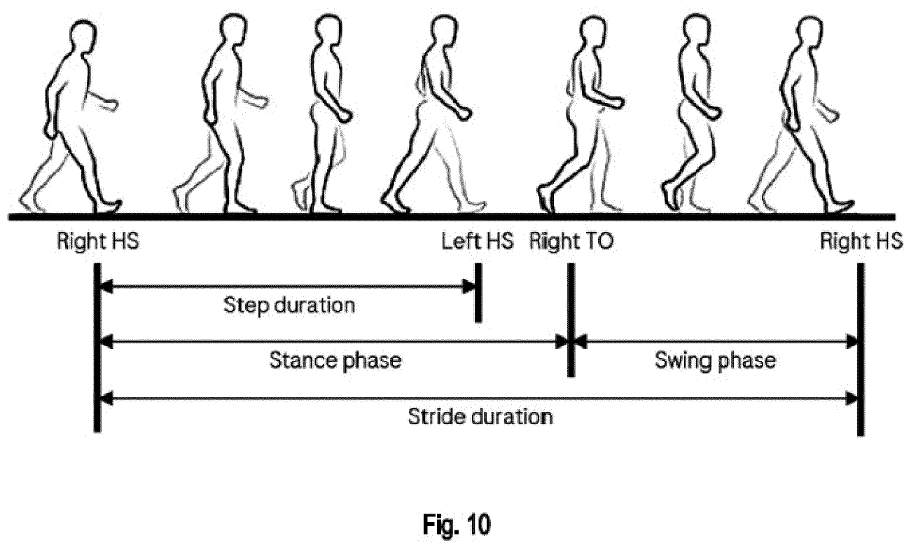

The invention relates to processing and/or statistical analysis of digital signals, for example to detect oscillatory patterns typical of gait activity. Gait activity as used herein refers to features of a plurality of gait cycles or fractions of a gait cycle. A gait cycle is a repetitive pattern consisting of a stance phase and a swing phase. The stance phase corresponds to the period of time when one foot is in contact with the ground. The swing phase corresponds to the period of time when one foot is not in contact with the ground. A human gait cycle is illustrated in FIG. 10. In normal human walking gait cycles, the stance phase counts for about 60% and the swing phase counts for about 40% of the gait cycle. The stance phase can be characterized by the following gait-cycle events for each foot: heel-strike (HS), foot-flat (FF), midstance, heel-off (HO), toe-off (TO). A HS event corresponds to the instant of initial contact of one foot with the ground. A FF event corresponds to the instant when the rest of the foot comes into contact with the ground. Midstance is defined when the center of mass is directly above the ankle joint center. A HO event corresponds to the instant when the heel begins to lift off the ground. A TO corresponds to the instant when the toe lifts off the ground. The swing phase for each foot begins with a TO and ends with a HS. Step length is the linear distance between the corresponding placements of right foot and left foot, for example the linear distance between a right-foot HS and a left-foot HS. Stride length is the linear distance between two successive placements of the same foot, i.e. two-step lengths, for example the linear distance between two successive right-foot HS. Step duration is the time used to complete the step length. Stride duration is the time used to complete the stride length, i.e. the full gait cycle duration equal to the stance phase duration plus the swing phase duration. Step frequency is inversely proportional to the step duration.

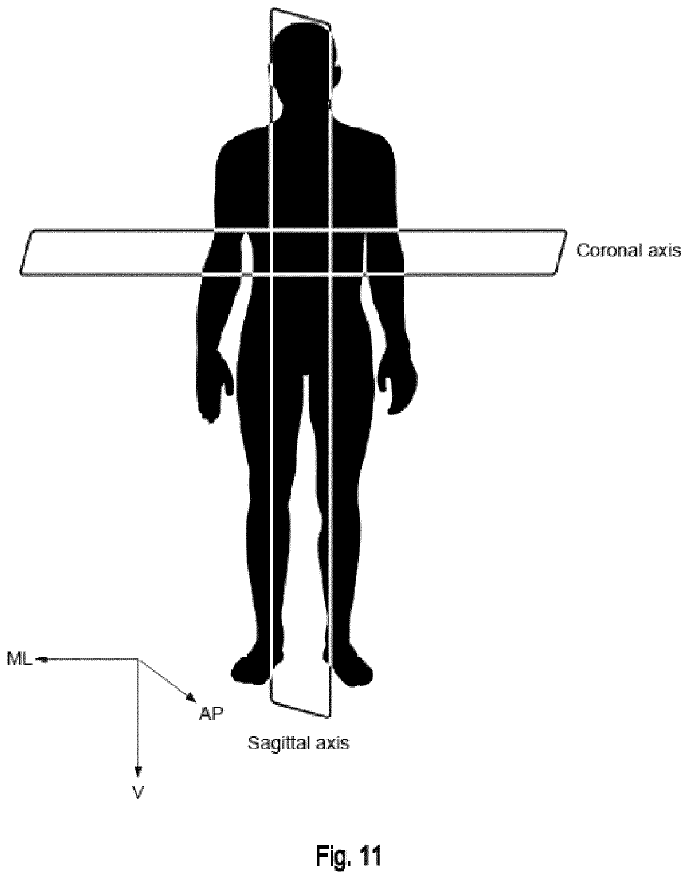

As used herein and as shown in FIG. 11, the antero-posterior component of the signal is the projection of the signal on the antero-posterior direction, i.e. along the sagittal axis x in the body reference system. As used herein and as shown in FIG. 11, the medio-lateral component of the signal is the projection of the signal on the medio-lateral direction, i.e. along the coronal axis y in the body reference system. As used herein and as shown in FIG. 11, the vertical component of the signal is the projection of the signal on the vertical direction, i.e. the vertical axis z in the body reference system, aligned with the direction of gravity.

As used herein, autocorrelation is the correlation of a signal with a time-shifted instance of itself. The autocorrelation or autocorrelation curve or autocorrelation sequence of a periodic signal has the same cyclic characteristics as the signal itself. As used herein, cross-correlation is the correlation of two independent signals. The cross-correlation or cross-correlation curve or cross-correlation sequence is a measure of the similarity of two signals as a function of the displacement of one relative to the other. As used herein, distances or displacements referred to in autocorrelations or cross-correlations are temporal distances or time shifts. In the present invention, the time-shifted instance of the signal is obtained by shifting the signal by one sample time step. The sample time step depends on the signal sampling frequency: if for example the signal sampling frequency is 50 Hz, then the sample time step is 0.02 seconds. The signal sampling frequency can be the sampling frequency of the wearable device. The signal sampling frequency can be the frequency at which the signal from the wearable device is resampled.

As used herein, continuous wavelet transforms or continuous wavelet transformations are intended in their established mathematical definition. As used herein, continuous wavelet functions are functions applied to the signal to obtain a continuous wavelet transform of the signal. As used herein, power spectral density is intended as the measure of a signal's power content. As used herein, a signal's power content is equivalent to a signal's frequency content. Power spectral densities are measured as a function of the signal frequency.

### Systems

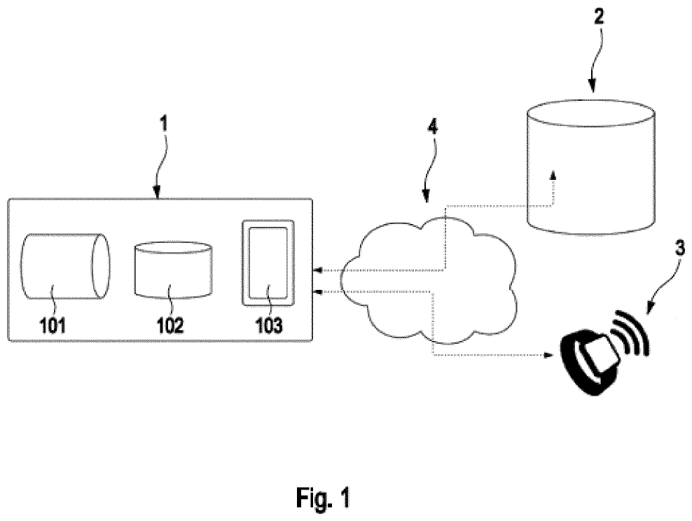

FIG. 1 illustrates an embodiment of a system that can be used to implement one or more aspects described herein. The system comprises a computing device 1, which comprises a processor 101 and a computer readable memory 102. In the embodiment shown, the computing device 1 also comprises a user interface 103, which is illustrated as a screen but can include any other means of conveying information to a user such as e.g. through audible or visual signals. The computing device 1 is communicably connected, such as e.g. through a network, to signal acquisition means, such as one or more wearable motion sensors, and/or to one or more databases 2 storing signals from the one or more wearable motion sensors. The one or more databases 2 can further store one or more of: control data, parameters (such as e.g. thresholds derived from control data, parameters used for normalization, etc.), clinical and/or patient related information, etc. The computing device can be a smartphone, tablet, personal computer or other computing device. The computing device can be configured to implement a method of processing signals associated with gait activity of a subject, as described herein. In alternative embodiments, the computing device 1 is configured to communicate with a remote computing device (not shown), which is itself configured to implement a method of processing signals associated with gait activity of a subject, as described herein. In such cases, the remote computing device can also be configured to send the result of the method of processing signals associated with gait activity of a subject. Communication between the computing device 1 and the remote computing device can be through a wired or wireless connection, and can occur over a local or public network 4 such as e.g. over the public internet. The signal acquisition means 3 can be in wired connection with the computing device 1, or can be able to communicate through a wireless connection, such as e.g. through WiFi and/or over the public internet, as illustrated. The connection between the computing device 1 and the signal acquisition means 3 can be direct or indirect (such as e.g. through a remote computer). The signal acquisition means 3 are configured to acquire signals from one or more wearable motion sensors located on a subject, for example accelerometer signals and/or gyroscope signals. In some embodiments, the acquired signals can have been subject to one or more preprocessing steps (e.g. normalizing, etc) prior to performing the methods described herein.

### Methods

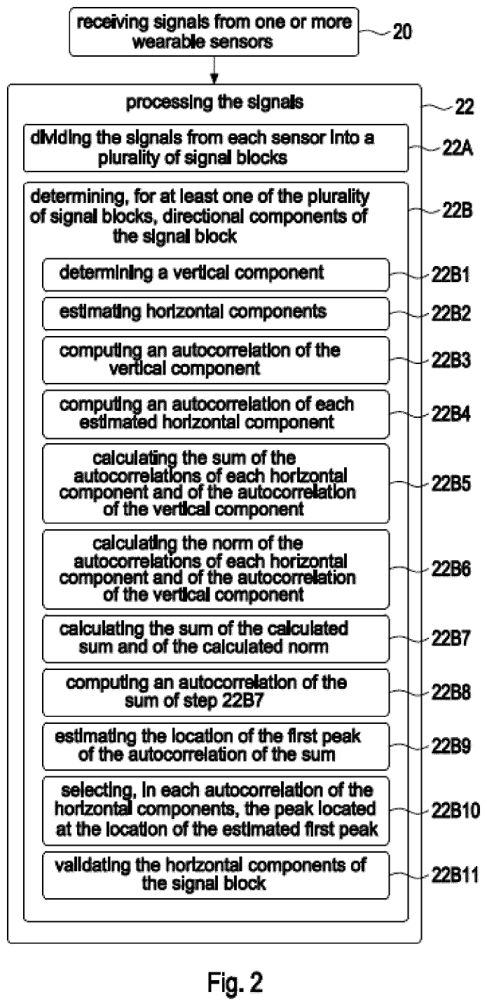

FIG. 2 is a flow diagram showing, in schematic form, a method of processing signals from one or more wearable motion sensors to analyse gait activity of a subject, according to the invention. With reference to FIG. 2, at step 20, signals from one or more wearable sensors are received. This comprises receiving accelerometer signals and gyroscope signals. Signals can be received directly from the sensors. Signals can have been previously recorded and received, for example from a memory or other computing devices. In an embodiment, signals can comprise measurements from an accelerometer at a plurality of time points. The measurements can comprise values of a measured instantaneous acceleration of the sensor relative to its frame of reference. The frame of reference of the sensor can be a frame of reference associated with the sensor. In an embodiment, signals can comprise measurements from a gyroscope at a plurality of time points. The measurements can comprise values of a measured instantaneous acceleration of the sensor relative to its frame of reference. The frame of reference of the sensor can be a frame of reference associated with the sensor. At step 22, the signals are processed. Processing can comprise several steps performed for each received signal, in the order as follows or in other orders. At step 22A, the signals from each sensor are divided into a plurality of signal blocks. For example, said signal blocks can be non-overlapping blocks and/or signal blocks of a predetermined duration. This step can comprise the step of classifying one or more of the plurality of signal blocks between a plurality of categories. The plurality of categories can comprise at least a rest category and a non-rest category. The non-rest category can comprise at least a gait category. The gait category can be referred to as straight-gait category. The gait category can encompass straight gait, semi-straight gait and curvilinear gait. Any gait that does not include turns above 90° can be referred to as “gait” or “straight-gait”. One or more of the plurality of blocks can be classified in one or more of the plurality of categories using a summarized metric over the block of the block's accelerator signal magnitude and/or of the block's gyroscope signal magnitude, and/or of the block's signal standard deviation. A summarized metric can be for example a mean, a median, trimmed versions thereof, maximum, minimum, percentile, suitably a median. The block's accelerator signal magnitude can be compared to a threshold that quantifies how close the magnitude is to the acceleration of gravity g. For example, the threshold can apply to the maximum difference between the accelerometer signal magnitude and the acceleration of gravity. For example, a threshold of 0.1 (or any other value x smaller than 0.5) may be used, such that the accelerometer signal magnitude is below the threshold if it is between 0.9 g (or (1−x)g) and 1.1 g (or (1+x)g)—where g is the acceleration of gravity. The acceleration of gravity g can be set equal to 9.81 m/s² with a precision of 2 decimals. Other choices of precision for the value of g can be made. When the accelerometer signal magnitude of the block is below the threshold (e.g. within a predetermined range around the acceleration of gravity), the block can be classified as a rest block. Conversely, when the accelerometer signal is at or above the threshold (e.g. outside of the predetermined range around the acceleration of gravity), the block can be classified as a non-rest block. The block's gyroscope signal magnitude can be compared to a threshold that quantifies the rotational speed of the sensor. For example, a suitable range of the gyroscope signal threshold can be between 0.2 rad/s and 0.6 rad/s. If a threshold of 0.2 rad/s is chosen, the tolerance for possible small movements is low. If a threshold of 0.6 rad/s is chosen, the tolerance for possible small movements is high. In particular, a low tolerance for small movements can be set in a Static Balance Test (SBT). In particular, a high tolerance for small movements can be set in a 2MWT (2-Minute-Walking-Test). When the gyroscope signal magnitude of the block is below the threshold, the block can be classified as a rest block. Conversely, when the gyroscope signal is at or above the threshold, the block can be classified as a non-rest block. The block's signal standard deviation can be compared to a threshold that quantifies the average spread of signal standard deviations around the block signal mean over the blocks within the same category. A suitable threshold can be the pooled standard deviation over the blocks within the same category. If the block's signal standard deviation is below said threshold, the block can be classified as a rest block. If the block's signal standard deviation is at or above said threshold, the block can classified as a non-rest block. For example a suitable range of the signal standard deviation threshold can be between 0.05 and 0.4, with 0.05 being the limit of rejecting static balance and 0.4 being a value tolerant of small movements. For example, a suitable threshold for the pooled standard deviation can be 0.2 m/s². Each signal block can be classified depending on one criterion, or a plurality of criteria. For example, if a signal block is classified as a rest block in two out of the three conditions on the three parameters, then the block is classified as a rest block. Alternatively, if the block is classified as a rest block in all three conditions on the three parameters, then the block is classified as a rest block. Alternatively, if the block is classified as a rest block in any one of the three conditions on the three parameters, then the block is classified as a rest block. In embodiments, a signal block is classified as a non-rest block only if the accelerometer signal magnitude of the block is at or above a predetermined threshold. This can be particularly useful when analysing signals collected by passive monitoring. Non-rest blocks can be further classified in straight-gait blocks and non-straight-gait blocks. Straight-gait blocks can be defined as blocks in which only movements in a single direction of motion are non negligible. For example, straight-gait blocks can be signal blocks recording a straight walking streak. Non-straight-gait blocks can be defined as signal blocks wherein movements in more than one direction of motion have non negligible magnitude. For example, non-straight-gait blocks can be signal blocks encoding a U-turn. Non-straight-gait blocks encoding a U-turn can be blocks wherein the gyroscope angular velocity is above a predetermined threshold. A suitable threshold can be 75 degrees per second. Non-straight gait blocks can be signal blocks encoding any turn with an angle above 90°. This can be referred to as “U-turn” for simplicity although U-turn blocks defined in this way would also encompass L turns. This step can comprise selecting one or more of a plurality of signal blocks classified as at least one of the plurality of categories. For example, signal blocks can be selected that are classified both as non-rest blocks and straight-gait blocks. Alternatively, signal blocks can be selected that are classified either as non-rest blocks, independently of whether they are straight-gait blocks, or as non-straight gait blocks. At step 22B, for at least one of the plurality of signal blocks, directional components of the signal block are determined. This step comprises the following steps, either in the following order or in another order: determining a vertical component (22B1), estimating horizontal components (22B2), computing an autocorrelation of the vertical component (22B3), computing an autocorrelation of each of the estimated horizontal components (22B4), calculating the sum of the autocorrelation of each horizontal component and of the autocorrelation of the vertical component (22B5), calculating the norm of the autocorrelation of each horizontal component and of the autocorrelation of the vertical component (22B6), calculating the sum of the calculated sum of autocorrelations and of the calculated norm of autocorrelations (22B7), computing an autocorrelation of the sum calculated in the previous step (22B8), estimating the location of the first peak of the autocorrelation of the sum (22B9), selecting in each autocorrelation of the horizontal components the peak located at the location of the first peak estimated in the previous step (22B10), validating the horizontal components of the signal block (22B11). Step 22B can further comprise a resampling of the signals to a predetermined sampling frequency. A suitable sampling frequency can be 50 Hz. At step 22B1, the vertical component can be obtained by applying an orientation filter to the signals. Any orientation filter known in the art can be used. For example, a Madgwick filter can be applied. At step 22B2, the horizontal components can be estimated by statistical analysis procedures, such as for example Principle Component Analysis (PCA). The directional components can be estimated via a sensor fusion algorithm. Steps 22B3 and 22B4 can further comprise filtering the autocorrelations to remove spurious peaks and obtain sinusoidal autocorrelations. For example, the signals can be filtered using a 2^{nd} order Butterworth low-pass filter. The filter can have a cut-off frequency to select the low-frequency spectrum, wherein harmonic frequencies related to gait activity mainly appear. A suitable cut-off frequency can be 6 Hz or lower, such as e.g. 3 Hz or 1 Hz. At step 22B5, the sum of the autocorrelations of the directional components is calculated as follows (Equation 1):

$$SUM = {auto}_{AP} + {auto}_{ML} + {auto}_{V}$$

wherein V stands for vertical, AP for antero-posterior, ML for medio-lateral. At step 22B6, the norm of the autocorrelations of the directional components is calculated as follows (Equation 2):

$$NORM = √ ( {auto}_{AP}^{2} + {auto}_{ML}^{2} + {auto}_{V}^{2} )$$

At step 22B7, the sum of the sum of autocorrelations and of the norm of autocorrelations is calculated as follows (Equation 3):

$$\sum = SUM + NORM$$

At step 22B8, the autocorrelation of the sum Z calculated at step 22B7 is performed. Step 22B8 can comprise a filtering of the autocorrelation to obtain a smoother curve. A suitable filter can be a Savitzky-Golay (savgol) filter. At step 22B9, the location of the first peak of the autocorrelation is estimated. The first peak is defined as the first maximum from the start of the autocorrelation curve. Step 22D9 can comprise removing peaks with a duration above a predetermined minimum. Step 22D9 can comprise removing peaks with a duration below a predetermined maximum. Step 22D9 can comprise removing peaks with a duration above a predetermined minimum and below a predetermined maximum. From the literature, a duration smaller than 0.3 seconds can correspond to a running pace. Therefore the predetermined minimum can be 0.3 seconds. From the literature, a duration longer than e.g. 1.1 or 1.5 seconds can correspond to a maximum step duration of a non-healthy subject. Therefore the predetermined maximum can be 1.1 seconds, or 1.5 seconds. At step 22B10, the location of the first peak obtained at step 22B9 is used to select the peaks in the autocorrelation curves at the same location estimated at step 22B9. At step 22B11, the selected peaks are used to validate the estimated horizontal components of the signal block. One or more predetermined criteria that apply to the signs of the selected peaks of each autocorrelation of the horizontal components are used to validate the estimated horizontal components. For example, the horizontal components can be correctly identified if: the selected peak of the antero-posterior component is positive and the selected peak of the medio-lateral component is negative; or the selected peak of the antero-posterior component and the selected peak of the medio-lateral component are negative and the selected peak of the antero-posterior component is the highest in absolute value. For example, the horizontal components can be incorrectly identified if: the selected peak of the antero-posterior component is negative and the selected peak of the medio-lateral component is positive; or the selected peak of the antero-posterior component and the selected peak of the medio-lateral component are negative and the selected peak of the medio-lateral component is the highest in absolute value. If at step 22B111 it is verified that the horizontal components are incorrectly identified, the two incorrectly identified components are swapped.

In embodiments, blocks identified as non-rest blocks can be further validated using data from a human activity recognition (HAR) model. For example, a block identified as a non-rest block can be validated by determining whether the output of a human activity recognition model for the block satisfies one or more predetermined criteria. A block that does not satisfy the one or more predetermined criteria can be excluded from further analysis, for example, analysis to extract gait features such as step and/or stride-based biomarkers. Note that this validation step can be performed at any point after identification of non-rest block, including after gait cycle events or steps have been detected, but before values of biomarkers derived from these are obtained (or even after values of biomarkers are obtained, where biomarker values associated with non-validated blocks can be excluded). Further, the human activity recognition (HAR) model can be applied to the signal before identification or rest and non-rest blocks. In other words, the methods can comprise comparing the results of a human activity recognition (HAR) model applied to the signal and the results of a step of classifying blocks as rest blocks and non-rest blocks as described herein, regardless of which of the human activity recognition model or block classification is applied first. Such comparisons can improve the accuracy of the biomarker values that are calculated. The one or more predetermined criteria can apply to the proportion or percentage of time in the block that is classified in one or more predetermined classes by the human activity recognition model. For example, as described further below, the HAR model can be configured to classify wearable motion sensor signal between a plurality of classes comprising one or more gait classes and one or more non-gait classes, and the one or more predetermined criteria can apply to the proportion or percentage of time in the block that is classified in a gait class by the human activity recognition model. As another example, as described further below, the HAR model can be configured to classify wearable motion sensor signal between a plurality of classes and the one or more predetermined criteria can apply to the proportion or percentage of time in the block that is classified in one or more predetermined classes of the plurality of classes by the human activity recognition model. In embodiments, the HAR model is configured to classify wearable motion sensor signal between a plurality of classes comprising one or more gait classes and one or more non-gait classes, and the one or more predetermined criteria comprise the proportion or percentage of time in the block that is classified in a gait class by the human activity recognition model being equal to or higher than a predetermined threshold. The predetermined threshold can be determined based on the particular circumstances, such as e.g. whether it is more desirable to stringently filter out any potentially mis-identified gait block or preserve as much potentially correctly identified gait block as possible. For example, a threshold of 50% can be used. Thus, the method can comprise applying a HAR model to a signal comprising a signal block to be assessed (e.g. a block that has been classified as a non-rest block using a method as described herein), and validating the block when the percentage of time in the block that is classified in a gait class by the human activity recognition model is equal to or higher than a predetermined threshold (e.g. 50%) or discarding the block when the percentage of time in the block that is classified in a gait class by the human activity recognition model is lower than the predetermined threshold.

A human activity recognition model (HAR) is a machine learning model trained to classify signals from one or more wearable motion sensors as described herein (i.e. signals from one or more wearable motion sensors located in any orientation on a subject, wherein the one or more wearable motion sensors comprise an accelerometer and a gyroscope) between a plurality of classes comprising at least one gait class and at least one non-gait class. The plurality of classes can comprise one or more gait classes and one or more non-gait classes. The machine learning model can be a deep learning model. The machine learning model can be a deep neural network comprising one or more convolutional layers and/or one or more recurrent neural network layers (such as e.g. one or more long short-term memory, LSTM, layers). The machine learning model can take as input accelerometer signal, such as e.g. signal along each of 3 orthogonal axes (e.g. time series of acceleration magnitude along the x, y, z axes). The one or more gait classes can comprise one or more classes selected from: walking, stairs, jogging. The one or more non-gait classes can comprise one or more classes selected from: sitting, standing and lying down. Alternative and/or additional classes can be defined depending on the circumstances (e.g. expected movement). The machine learning model can be trained using annotated labels for periods of movement corresponding to the activities in the respective classes of the plurality of classes (ground truth class). An example of a HAR model that can be used in the context of the present disclosure is described in Cheng et al. (Human Activity Recognition from Sensor-Based Large-Scale Continuous Monitoring of Parkinson's Disease Patients, 2017 IEEE/ACM International Conference on Connected Health: Applications, Systems and Engineering Technologies (CHASE)).

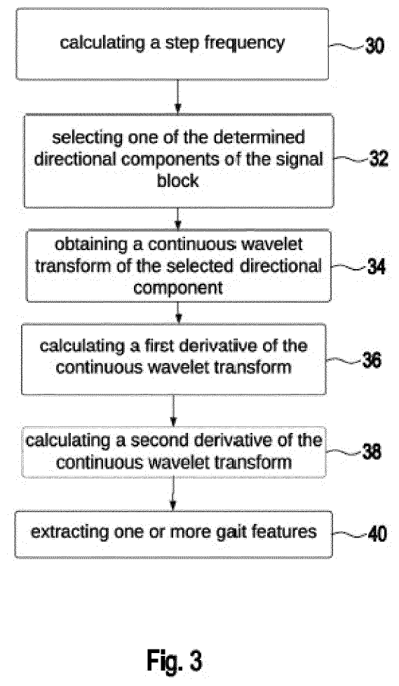

FIG. 3 is a flow diagram showing, in schematic form, a method of extracting one or more gait features, according to the invention. This method can be performed after the method of FIG. 2. With reference to FIG. 3, at step 30 a step frequency is calculated for at least one of the one or more signal blocks, selected as described in FIG. 2. The step frequency is calculated using the directional components of the signal block, determined as described in FIG. 2. At step 32, one of the determined directional components of the signal block is selected using the calculated step frequency and the autocorrelations of each directional component, computed as described in FIG. 2. At step 34, a continuous wavelet transform of the selected directional component is obtained. A general mathematical expression of a continuous wavelet transform is the following (Equation 4):

$${X}_{ω} ( a , b ) = \frac{1}{{\vert a \vert}^{1 / 2}} {\int }_{- ∞}^{∞} x ( t ) \overline{ψ} ( \frac{t - b}{a} ) dt$$

wherein x(t) corresponds to the selected directional component, yr is the continuous wavelet function, t is the time, a and b are parameters. At step 36, a first derivative of the continuous wavelet transform is calculated. This step allows to identify heel-strike events as the minima of the first derivative. At optional step 38, a second derivative of the continuous wavelet transform is calculated. This step allows to identify toe-off events as the maxima of the second derivative. In another embodiment, toe-off events are the minima of the first derivative and heel-strike events are the maxima of the second derivative. At step 40, one or more gait features can be extracted. For example, gait features such as the timing and/or location of the gait-cycle events, and in particular heel-strike events, and/or of the toe-off events can be extracted. Other gait features can be extracted from the heel-strike events and optionally the toe-off events, such as for example cadence, fatigue, stability, rhythm/variability, asymmetry, pace, forward balance, lateral balance. Step 40 can further comprise a refinement of the gait feature extraction. For example, false positive (heel-strike or toe-off) events can be removed. This can be done by setting predetermined thresholds, for example on the stride duration. Alternatively, missing gait-cycle events or extra gait-cycle events can be corrected for by leveraging the expected order of gait-cycle events. For example, previous gait cycles can be used to determine the most likely position of gait-cycle events in subsequent gait cycles.

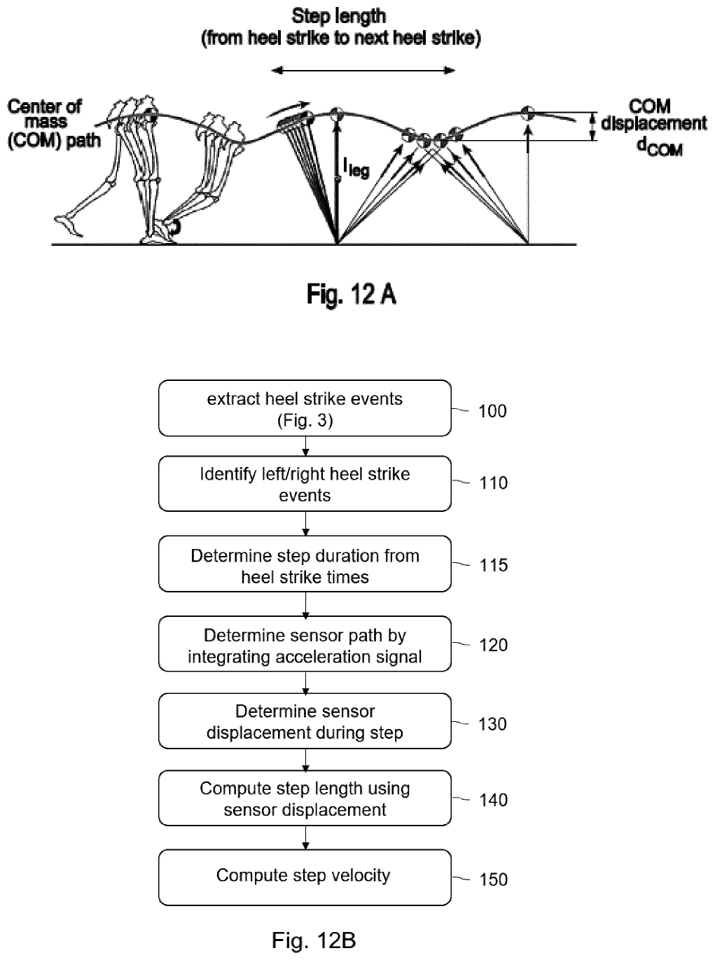

The identified gait-cycle events can further be used to quantify the values of one or more gait features, such as gait features that can be used as biomarkers as described herein. In embodiments, the one or more gait features comprise a step velocity, stride velocity, or statistic derived therefrom (such as e.g. mean, percentile, quartile, etc. of a set of step or stride velocity values). FIG. 12B is a flow diagram showing, in schematic form, a method of calculating the step velocity of a subject, according to the invention. The step velocity for a step can be defined as the ratio of the step length to the step duration. The stride velocity for a stride can be defined as the ratio of the stride length to the stride duration. Reference to a step velocity encompasses a left step velocity (where a left step is characterize as lasting from a right heel strike to a left heel strike), a right step velocity (where a right step is characterized as lasting from a left heel strike to a right heel strike), and a step velocity calculated for any step characterized as lasting from a heel stroke to the next heel strike (where the next heel strike is a contralateral heel strike). This is illustrated on FIG. 10. A stride includes two consecutive steps (a left step and a right step). A stride can therefore be characterized as lasting from a right heel strike to the next subsequent right heel strike, or from a left heel strike to the next subsequent left heel strike. A full gait cycle includes two heel strikes. Thus, a step or stride duration can be straightforwardly calculated from the timings of identified gait cycle events (step 115). Calculating a step length or stride length can be performed using any method known in the art. For example, the methods described in Soltani et al. 2021 can be used. In embodiments, the calculating of a step length (or stride length as the combined length of two consecutive steps) can be performed using biomechanical modelling and anthropometric measures as will now be described. In particular, the acceleration signal from a wearable motion sensor can be integrated twice to determine a path of the sensor (step 120). A path is a set of positions as a function of time. The displacement of the sensor during a step can then be determined (step 130) from this path (d_{COM}) as difference between the maximum and minimum of the path during the step (i.e. between two consecutive previously identified heel strikes), as illustrated on FIG. 12A. The step length can then be determined from this (step 140) as follows (Equation (6)):

step length=2\sqrt{2l_{leg}d_{COM}−d_{COM}²} where d_{COM} is the displacement of the sensor during the step and l_{leg} is the distance of the sensor from the floor (which can be equal to the length of the leg of the subject when the sensor is located in the vicinity of the subject's centre of mass, such as e.g. in the waist, lumbar or hip region). The distance of the sensor from the floor can be provided as a parameter to the method. The distance of the sensor from the floor can be set to a user received value (e.g. a previously measured leg length if the wearable sensor is located around the waist) or can be estimated from the subject's height in case the location of the wearable sensors is known. The acceleration signal used can be the acceleration along the vertical axis (vertical component of the acceleration signal), in which case the displacement of sensor calculated is a vertical path displacement. Alternatively, the signal used can be the magnitude of the acceleration signal, in which case the displacement of the sensor calculated is a magnitude of sensor displacement. Equation (6) can be used to determine the step length in either case. Use of the acceleration magnitude rather than vertical acceleration advantageously accounts for lateral displacement, thereby making the length estimations more robust. The step of determining the path by integrating the sensor signal twice can comprise one or more filtering and/or detrending steps prior to the first and/or second integration. Detrending a signal refers to removing a linear component that may be present in the signal. This can be performed using any method known in the art, see e.g. Python function scipy.signal.detrend( ). Filtering a signal can also be referred to as smoothing and can use any filter known in the art, such as a high pass filter like a Butterworth filter (e.g. 4^{th} order Butterworth filter with a cutoff of 1 Hz). In embodiments, the step of determining the path by integrating the sensor signal twice can comprise detrending the signal prior to the first integration and filtering the integrated signal prior to the second integration. The steps of determining the path, the path displacement and the step length can be performed for a single step (e.g. using signal from a first identified heel strike to the next identified heel strike), a single stance (from heel strike to toe-off of the same leg), a single stride (e.g. using signal from a first identified heel strike to next identified heel strike on the same side, i.e. the second next identified heel strike), or for a plurality of steps, stances or strides. When determining the path for a plurality of steps, the acceleration signal can be integrated iteratively for each consecutive step by, at each iteration, using the velocity (for the first integration, respectively the position, for the second integration) estimated at the previous iteration as an initial condition for the numerical integration. This resets the initial condition for the integration(s) at every step, ensuring that numerical errors do not propagate and increase along the estimated path. Note that this does not assume a specific velocity as an initial condition for the integration as one may not be known. Indeed, contrary to situations in which the sensor is located e.g. on the foot (in which case the velocity can be assumed to be 0 at the start of a step when the foot is on the floor), when the sensor is located away from the foot (such as e.g. near the centre of mass) there is no point in the gait cycle where a specific predetermined velocity can be assumed. This poses additional challenges for accuracy of the step length detection, which are solved by the above-described method. In other words, if the sensors are on the feet, the step length can be calculated more accurately because the assumption of zero velocity is always satisfied when the heel touches the floor (the velocity is always zero when the foot touches the floor). If we move far away from the feet this assumption is not valid anymore. However the above provides a solution to that problem by resetting the status at each iteration both for the velocity and for the position. Finally, the determined step length and step duration can be used at step 150 to determine a step velocity as the ratio of these two values (step length/step duration), or a stride velocity as the ratio of a stride length (combined length of two consecutive steps) and stride duration. Note that the determination of the step duration is independent of the determination of the step length and can be performed at any point after identification of heel strike events.

In embodiments, step and/or stride analysis is performed without differentiating between left and right events (i.e. simply identified any heel strike as a heel strike, where the phase between two consecutive heel strikes is a step). Alternatively, any heel strike even can be identified as a left or right strike event at optional step 110. This can be performed using a method as described in McCamley et al. (2012). In particular, signals received from a gyroscope forming part of the one or more wearable motion sensors (e.g. a sensor located around the waist/lumbar area of the subject can be used) can be used to differentiate between left and right heel strikes based on the sign of the angular velocity around the vertical axis determined from the gyroscope signal (optionally after filtering the signal for example using a Butterworth filter, such as a fourth order Butterworth filter). In particular, when the sensor is oriented such that the vertical direction is negative, a positive peak in this signal can be associated with a left heel strike and a negative peak in this signal can be associated with a right heel strike event. Similarly, when the sensor is oriented such that the vertical direction is positive, a positive peak in this signal can be associated with a right heel strike and a negative peak in this signal can be associated with a left heel strike event Thus, a method as described herein can comprise obtaining, from the gyroscope signal, a signal corresponding to the vertical angular velocity of the sensor (vertical component of the gyroscope signal), optionally smoothing said signal (e.g. using a Butterworth filter or the like), and determining whether a previously identified heel strike event is a left heel strike event or a right heel strike event based on the sign of said (optionally smoothed) signal at the time of the identified heel strike event. In particular, a heel strike event can be identified as a left heel strike event when the (optionally smoothed) signal is positive at the time of the heel strike event, and a heel strike event can be identified as a right heel strike event when the (optionally smoothed) signal is negative at the time of the heel strike event.

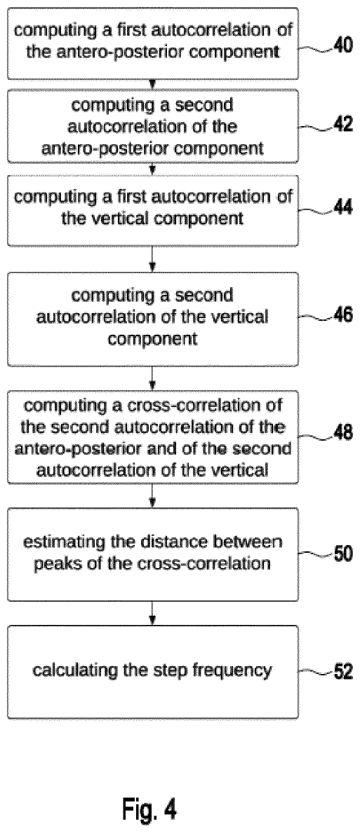

FIG. 4 is a flow diagram showing, in schematic form, a method of calculating the step frequency of a subject, according to the invention. This method can be performed after the method of FIG. 2. This method can implement step 30 of FIG. 3. With reference to FIG. 4, at step 40 a first autocorrelation of the antero-posterior component is computed (auto_{AP}). At step 42, a second autocorrelation of the antero-posterior component is computed (auto2_{AP}). The second autocorrelation is computed as the autocorrelation between the first autocorrelation of the antero-posterior component and a time-shifted version of the first autocorrelation of the antero-posterior component (auto2_{AP}=autocorrelation(auto_{AP}, auto_{AP}(t)), where auto_{AP}(t) is a time-shifted version of auto_{AP}). At step 44, a first autocorrelation of the vertical component is computed (autoᵥ). At step 46, a second autocorrelation of the vertical component is computed (auto2ᵥ). The second autocorrelation is computed as the autocorrelation between the first autocorrelation of the vertical component and a time-shifted version of the first autocorrelation of the vertical component (auto2ᵥ=autocorrelation(autoᵥ, autoᵥ(t)), where autoᵥ(t) is a time-shifted version of autoᵥ). Steps 44 and 46 can be performed before steps 40 and 42. At step 48, a cross-correlation of the second autocorrelation of the antero-posterior component and of the second autocorrelation of the vertical component is computed. The signal can be an accelerometer signal and the cross-correlation can be a cross-correlation of the second autocorrelation of the antero-posterior component of the accelerometer signal and of the second autocorrelation of the vertical component of the accelerometer signal. Alternatively, the signal can be a gyroscope signal and the cross-correlation can be a cross-correlation of the sum of the second autocorrelation of the antero-posterior component of the gyroscope signal and of the second autocorrelation of the vertical component of the gyroscope signal. At step 50, the distance between peaks of the cross-correlation is estimated. The distance between every peak (e.g. when using accelerometer signal) or every other peak (e.g. when using gyroscope signal) contains information about the stride duration. The stride duration can be computed via a summarized metric of the distance between every peak or every other peak. Said summarized metric can be selected from mean, median, moving average, and trimmed versions thereof. In embodiments, the summarized metric can be computed as a summarized metric of the distance between every other peak that satisfy a predetermined criterion (e.g. distance below a predetermined maximum distance). The distance between every peak or every other peak contains information about the step duration or stride duration. For example, when using accelerometer signal the distance between every other peak contains information about step duration. When using gyroscope signal the distance between every peak contains information about the stride duration. The step or stride duration can be computed via a summarized metric of the distance between every peak or every other peak. Said summarized metric can comprise mean, median, moving average, trimmed versions thereof. In embodiments, the cross-correlation signal may be smoothed using a moving average prior to determining the distance between peaks of the cross-correlation at step 50. At step 52, the step frequency is calculated from the estimated distance between peaks. In an embodiment, the step frequency is calculated as the half of the inverse of the stride duration. In another embodiment, the step frequency is calculated as the inverse of the step duration. The step frequency calculated from the stride duration and the step frequency calculated from the step duration shall lead to a similar result within a certain precision. In another embodiment, the antero-posterior component is replaced by the medio-lateral component. This can be useful for example when analyzing signals from a U-Turn test.

The calculation of a step frequency using the determined directional components of the signal block can be performed using determined directional components from received signals from the accelerometer comprised in the one or more wearable motion sensors. Instead or in addition to this, the calculation of a step frequency using the determined directional components of the signal block can be performed using determined directional components from received signals from the gyroscope comprised in the one or more wearable motion sensors. The present inventors have found that both the gyroscope signal and the acceleration signal could be used to determine a step frequency as described herein. The present inventors have further found that the use of the gyroscope signal was particularly advantageous as it tends to be more stable in practice, therefore enabling more robust estimation of the step frequency. In embodiments using the accelerometer signal, the step frequency can be identified as the frequency which corresponds to the first peak in the cross-correlation signal provided that the first peak is within a predetermined distance of the origin of the signal corresponding to half of the summarized metric of the distance between every other peak. In embodiments using the gyroscope signal, the step of computing a cross-correlation of the second autocorrelation of the antero-posterior component and of the second autocorrelation of the vertical component comprises computing a cross-correlation of the sum of the second autocorrelation of the antero-posterior component and of the second autocorrelation of the vertical component. In embodiments using the gyroscope signal, the distance between peaks in this signal contains information about the stride duration. The stride duration can be computed via a summarized metric of the distance between every peak. Said summarized metric can be selected from mean, median, moving average, and trimmed versions thereof. The step frequency can be calculated as half of the summarized distance between peaks. The present inventors have recognized that the gyroscope signal is characterized by having a quasi-periodic behavior resulting from gait motion, and that the cross-correlation between the sum of the second antero-posterior and vertical autocorrelations of the gyroscope signal amplifies this periodicity, thereby enabling robust detection of stride frequency. The step frequency can be assumed to be half of the stride frequency, in turn enabling robust detection of step frequency.

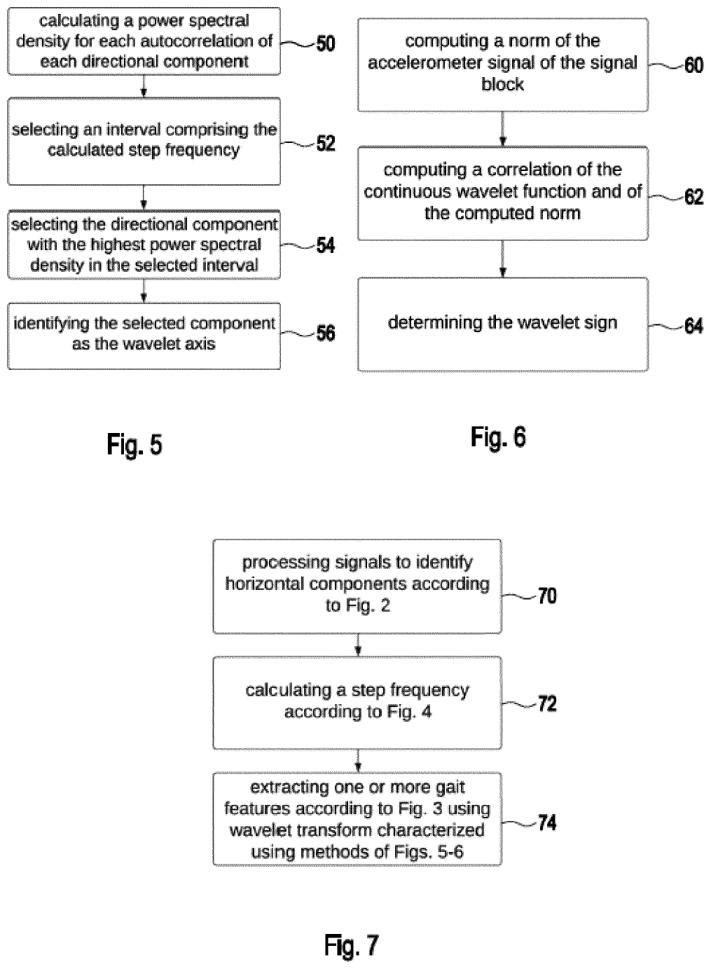

FIG. 5 is a flow diagram showing, in schematic form, a method of selecting the wavelet axis of a continuous wavelet function, according to the invention. This method can be performed after the method of FIG. 2. This method can implement step 34 of FIG. 3. With reference to FIG. 5, at step 50 a power spectral density for each autocorrelation of the directional components is calculated. A general mathematical expression of a power spectral density is the following (Equation 5):

$$PSD ( f ) = {lim}_{T \to ∞} \frac{1}{T} {\vert {\hat{x}}_{T} ( f ) \vert}^{2}$$

where t is the time, T is a time period, \hat{x}ᵣ(f) is the Fourier transform of x_{T}(t)=x(t)ωr_{T}(t), where x(t) is the autocorrelation of a directional component and ω_{T}(t) is a function equal to 1 within the time period T and 0 elsewhere.

At step 52, an interval is selected comprising the calculated step frequency. The interval is a frequency (or power) interval. The interval can be defined as a range of values within +/−25% (or within +/−10%, +/−15%, +/−25%, or +/−30%) of the calculated step frequency, such as e.g. values at or above S−0.25S and at or below S+0.25S, where S is the step frequency. At step 54, the directional component with the highest power spectral density in the selected interval is selected. At step 56, the selected directional component is identified as the wavelet axis. The wavelet axis defines which directional component of the signal undergoes the continuous wavelet transform.

FIG. 6 is a flow diagram showing, in schematic form, a method of estimating the wavelet sign of a continuous wavelet function, according to the invention. This method can be performed after the method of FIG. 2. This method can implement step 34 of FIG. 2. With reference to FIG. 6, at step 60, a norm of the accelerometer signal block is computed (|acc|). At step 62, a correlation of the parameterized continuous wavelet function and of the calculated norm is computed. In an embodiment the correlation is computed via a Pearson coefficient. At step 64, the wavelet sign is determined. The wavelet sign can be determined based on the sign of the computed cross-correlation. If the sign of the computed correlation is positive, then the wavelet sign is positive. If the sign of the computed correlation is negative, then the wavelet sign is negative. In an embodiment, if the correlation is zero, then the correlation of the norm of the accelerometer signal block and of the medio-lateral gyroscope signal block can be computed. If the latter correlation is negative, the wavelet sign is positive. If the latter correlation is positive, the wavelet sign is negative. Step 62 can comprise a filtering of the antero-posterior component. For example, the signals can be filtered using a 2^{nd} order Butterworth low-pass filter. The filter can have a cut-off frequency to select the low-frequency spectrum, wherein harmonic frequencies related to gait activity mainly appear. A suitable cut-off frequency can be 2 Hz. Step 62 can comprise a filtering of the correlation. If the correlation is below a predetermined minimum threshold, the correlation is truncated to zero. Experimental results show that said predetermined minimum threshold can be below 0.3.

FIG. 7 is a summary figure showing the methods of the previous figures relative to each other, according to the invention. At step 70, signals are processed to identify horizontal components according to FIG. 2. At step 72, a step frequency is calculated according to FIG. 4. At step 74, one or more gait features are extracted according to FIG. 3 using a wavelet transform characterized using methods of FIGS. 5-6.

### Examples

The examples below illustrate applications of the methods of the present invention, in particular in the context of analyzing signals associated with gait activity of a subject for the purposes of assessing symptoms of MS. The signals processed and analysed in the following examples were received from subjects undergoing several tests in two on-site visits in a gait laboratory and during an unsupervised remote testing period for 10-14 days in between the on-site visits. The participants performed several gait and balance tests, including a 2MWT (2-Minute-Walking-Test), a U-Turn Test, and a SBT (Static Balance Test). The 2MWT comprised four separate conditions, or walking tasks, each lasting two minutes: 1) fixed speed (2 km/h), 2) self-paced (at normal pace), 3) fast-paced (as fast as the subject can walk safely), and 4) dual-task (self-paced condition with a simultaneous cognitive task consisting of serial subtractions of 7 starting from 200). The U-Turn Test instructed the participants to walk back and forth for 60 seconds while performing U-turns roughly 3 or 4 m apart. The SBT battery consisted of five test conditions, each performed twice and lasting 30 seconds: 1) natural stance with feet apart and eyes open, 2) natural stance with feet apart and eyes closed, 3) parallel stance with feet together and eyes open, 4) full tandem stance with eyes open, 5) single foot stance with eyes open. The participants carried smartphones in six different locations: right and left front pockets, central front at the waist, left and right back pockets, back waist.

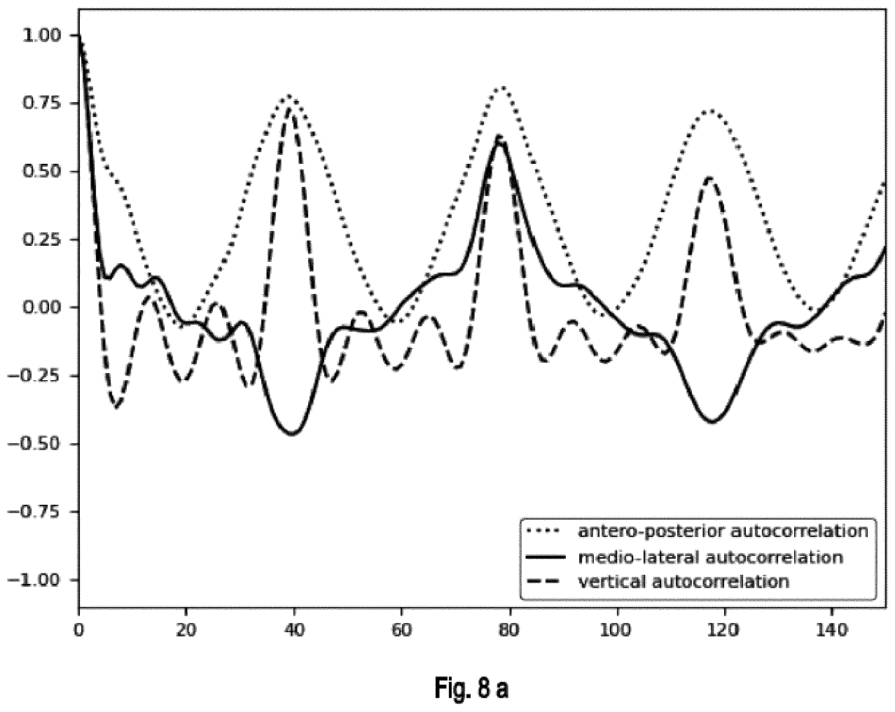

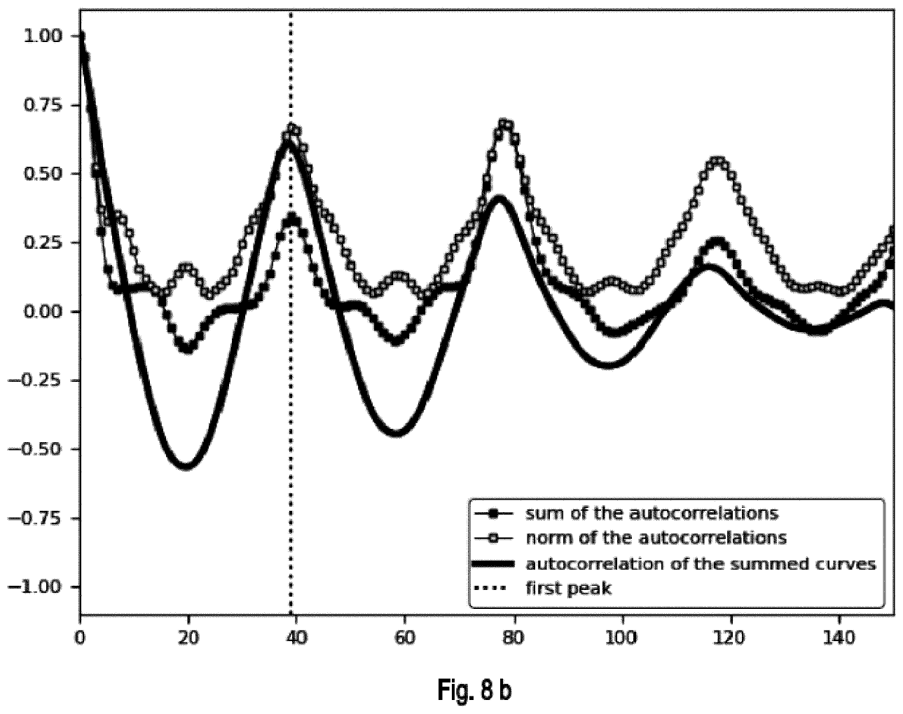

FIG. 8 shows an example of a method of processing signals from one wearable motion sensor to analyse gait activity of a subject, according to the invention. The signals received were divided in signal blocks and resampled to a sampling frequency of 50 Hz, i.e. each sample duration was 0.02 seconds. The three directional components, i.e. vertical, antero-posterior, and medio-lateral, were obtained by applying a Madgwick filter to the 3D signals and a PCA. FIG. 8a shows the autocorrelations of the three directional components, i.e. antero-posterior, medio-lateral and vertical components, of the signals selected in a straight-gait block obtained from a wearable sensor on a subject. FIG. 8b shows the sum of the three autocorrelations, the norm of the three autocorrelations, and the autocorrelation of the summed sum and norm. The first peak of the autocorrelation of the summed curves, marked by a vertical line, was used to estimate the step duration. In this example, the estimation of the step duration was slightly below 40 samples, i.e. 0.8 seconds. The estimated step duration was used to validate the directional component curves according to the invention, namely: the peak located at around 40 samples in the antero-posterior component was positive and the peak located at around 40 samples in the medio-lateral component was negative, thus confirming that the antero-posterior component and the medio-lateral components were correct as identified through the Madgwick filter and PCA. In the literature, the three directional components are obtained by projecting the signals on the global walking directions assumed by a predefined frame of reference. With the computer-implemented method of the invention as hereinbefore described, the present inventors were able to first estimate the directional components and then confirm the estimated directional components without the need for assuming a predefined frame of reference.

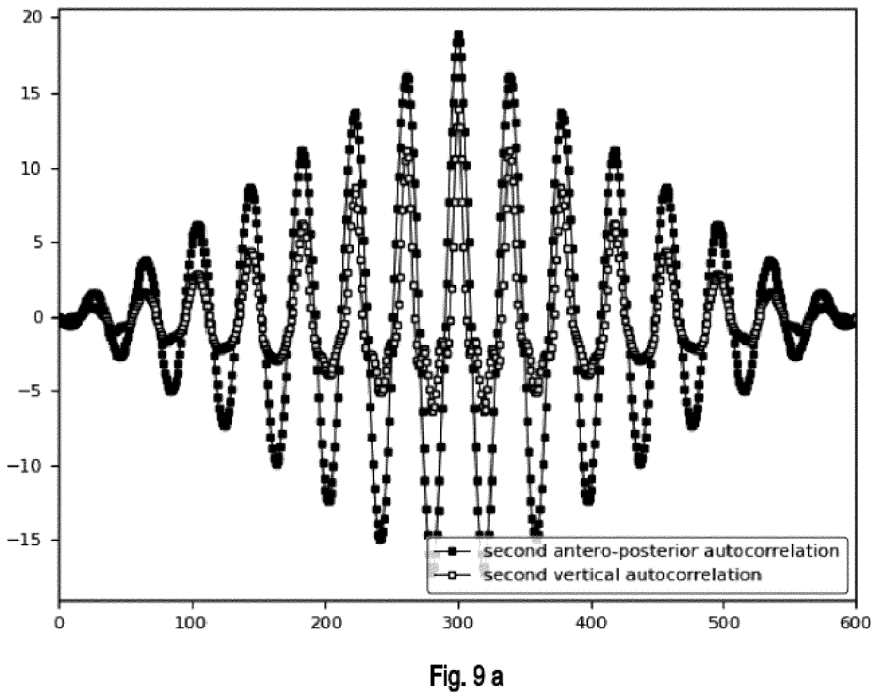

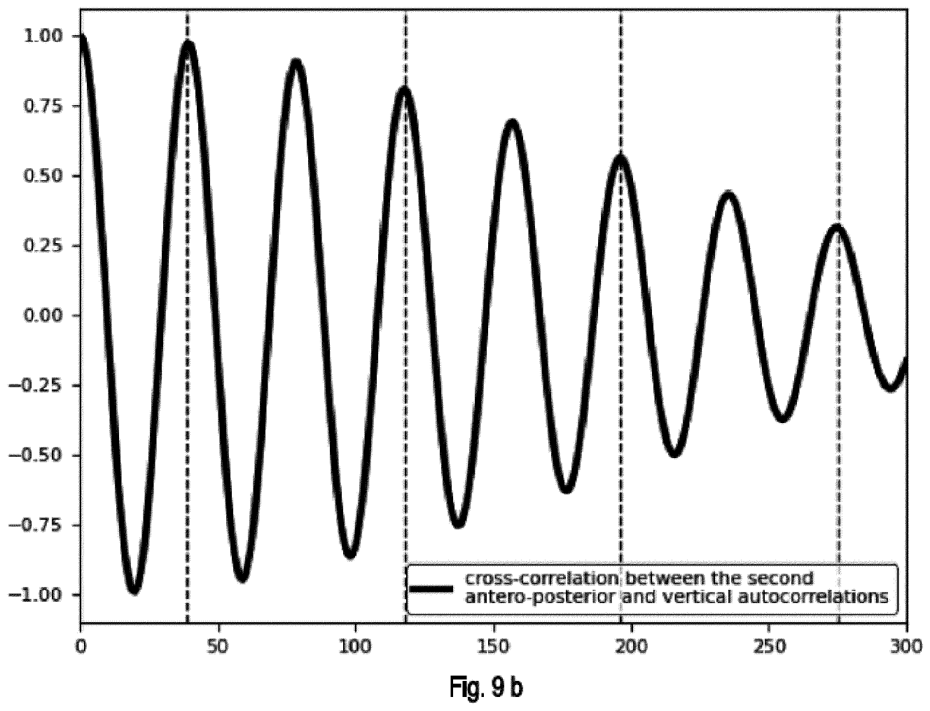

FIG. 9 shows an example of a method of calculating the step frequency of a subject, according to the invention. FIG. 9a shows the second autocorrelations of two directional components, in particular the antero-posterior and vertical components. FIG. 9b shows the cross-correlation of the two curves in FIG. 9a. Starting from the first peak at around 40 samples, every other peak is marked by a vertical line. The distance between the vertical lines quantifies the stride duration. An aggregate estimate of the stride duration was obtained by calculating the median of the distances between the vertical lines, i.e. between every other peak. A peak-finding function, i.e. a function that takes in input the stride duration and outputs the position of the peaks, was used to find the peaks in the cross-correlation curve. In this example, a further validation step was performed at this point: it was verified that a peak existed at a location of roughly half of the stride duration in the autocorrelation of the directional components. If this was the case, then the stride duration estimated was considered reliable, otherwise the peak-finding function was recalculated with an updated input, for example the stride duration increased by 0.02 seconds, until the new stride duration could be considered reliable. The estimated step frequency was then calculated as half the inverse of the stride duration. In this example, the estimated stride duration was circa 80 samples (1.6 seconds), and the estimated step frequency was 1.25 Hz. In the literature, a step frequency is estimated directly from the peaks of the autocorrelations. However, such method is not robust enough in comparing signals from different patients or different sensor locations and/or orientations. The present inventors were able to use the more robust estimation of the step frequency according to the invention to extract gait features comparable across subjects. The present inventors were also able to use the more robust estimation of the step frequency according to the invention to extract gait features comparable across sensor locations and/or orientations. Additionally, in the literature the wavelet scale of the continuous wavelet function used for analyzing the signals is either set as a constant (McCamley et al, 2012; Godfrey et al, 2014; Del Din et al, 2015) or set as proportional to a constant step frequency (Ning et al, 2019; Zhou et al, 2016), or chosen via a regression model (Caramia et al, 2019) or as the most dominant frequency of the power spectral density (Pham et al, 2017). The present inventors were able to use the more robust estimation of the step frequency according to the invention to precisely calculate the wavelet scale of the continuous wavelet function, which in turns results in more accurate gait features.

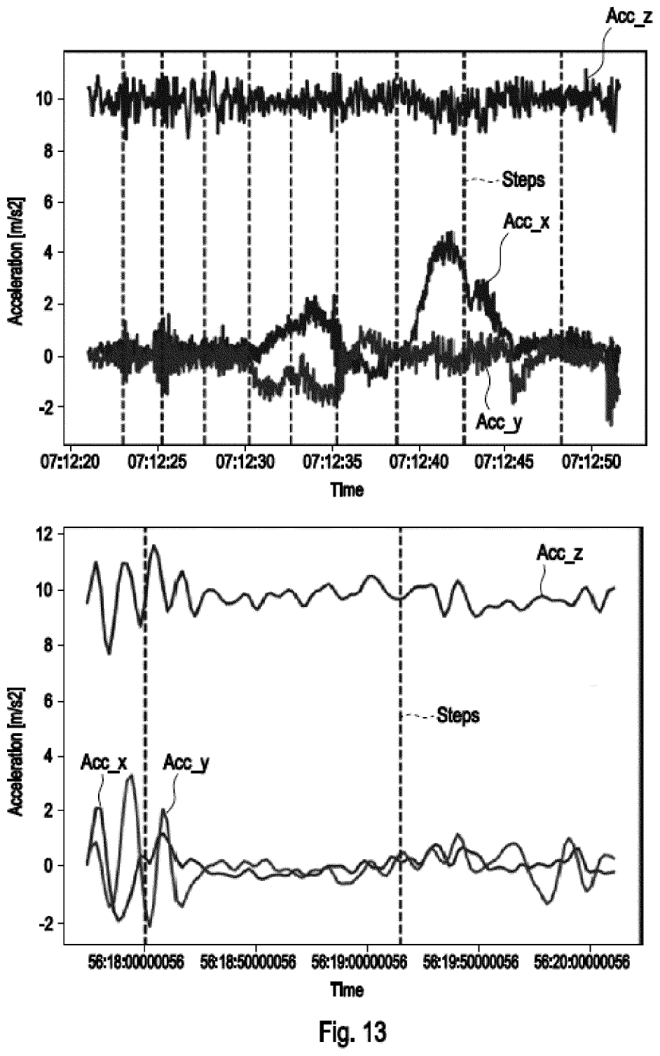

FIG. 13 shows example results of an embodiment of a method of processing signals from one or more wearable motion sensor to analyse gait activity of a subject, according to the invention, in which a single threshold on magnitude of the signal received from the accelerometer was used to classify blocks as rest vs non-rest blocks. In particular, a signal block was classified as a non-rest block only if the accelerometer signal magnitude of the block was outside of a predetermined range around the acceleration of gravity, in particular 0.9 g to 1.1 g where g is the acceleration of gravity which was set equal to 9.81 m/s². FIG. 13 shows two examples of signal blocks that were excluded from analysis for passive monitoring based on the acceleration magnitude criteria, but that satisfied at least one of the gyroscope magnitude and acceleration standard deviation criteria, and from which steps would have otherwise been erroneously inferred. This approach is particularly useful in the context of passive monitoring (e.g. for the determination of biomarkers such as step or stride velocity and metrics derived therefrom), where large amounts of data may be available and it is more important to remove false positive gait bouts than risk losing false negative gait bouts. The present inventors have found that applying this particular filtering approach resulting in the exclusion of false positive gait bouts, ultimately resulting in more accurate step or stride velocity estimates obtained from passive monitoring data. In other contexts such as when using signals from other motion tests, the use of criteria that apply to other signals (such as gyroscope magnitude and/or acceleration standard deviation as described above) instead or in addition to the above can also be useful. In other words, this embodiment is particularly useful for the detection of step and stride parameters from passive monitoring signals, but all other embodiments describe remain usable and can be advantageous in other situations.

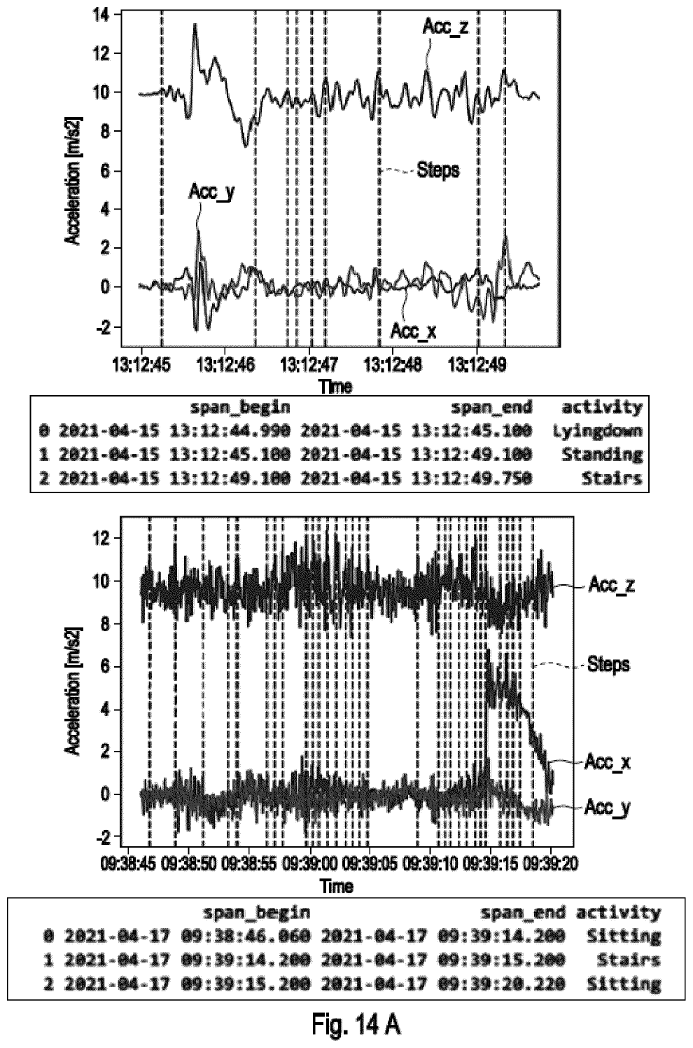

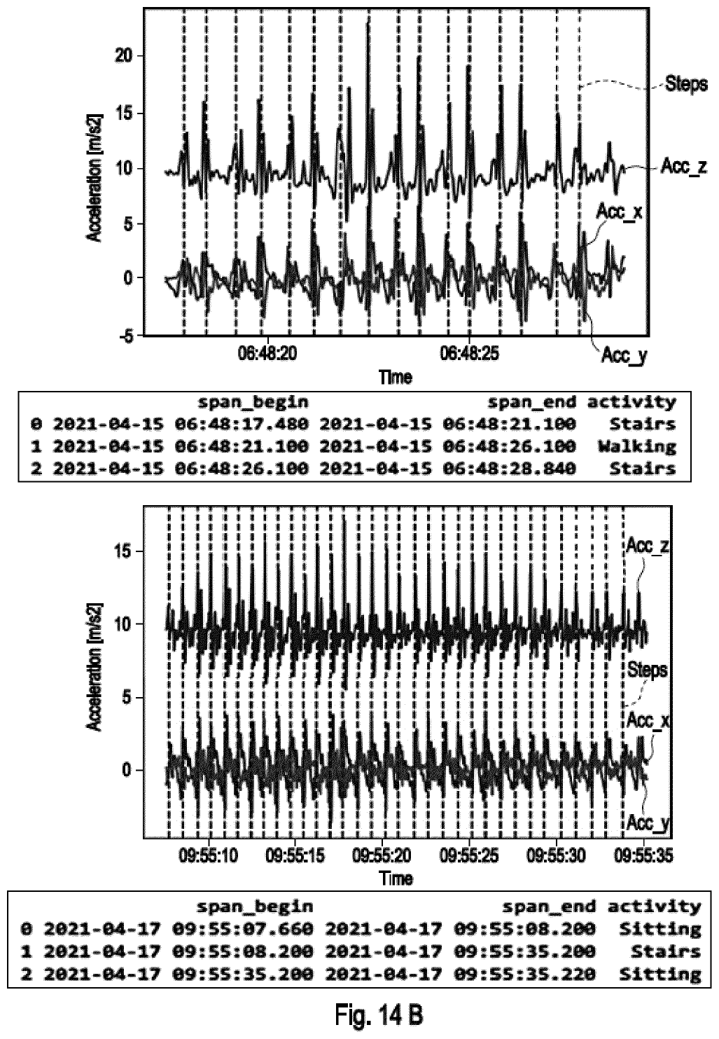

FIG. 14 shows example results of an embodiment of a method of processing signals from one or more wearable motion sensor to analyse gait activity of a subject, according to the invention, in which identified non-rest blocks were further validated using information from a human activity recognition model. In particular, gait bouts (non-rest blocks, in which step detection would normally be applied) were merged with information from the Human Activity Recognition (HAR) model to evaluate correctness of the detection and validation of steps while walking. The HAR model was implemented as described in Cheng et al. (2017). For each block, the total percentage of the amount of time in the block spent in activities detected as gait activities (here defined as walking, stairs, jogging) by the HAR model was calculated (i.e. (Time in gait activities/total time)×100). A block was excluded if the calculated % was below 50%, and maintained if the calculated % was at or above 50%. This post-processing approach further improves the quality and reliability of the data used to calculate step/stride related biomarkers, by discarding or validating steps while walking in blocks that can have passed a threshold on the acceleration magnitude but can contain activity that is not in fact the result of the subject performing gait cycles. FIG. 14 A shows two examples of incorrectly detected steps while walking, that are discarded once the post-processing step using HAR is applied as the acceleration bout does not contain gait-related information (signals and detected steps are shown in the top panel with the HAR model output underneath, for each of the two examples). Those steps were removed from the analysis performed to obtain values for step/stride-based biomarkers. FIG. 14B shows two examples of correctly detected steps while walking, that are validated once the post-step using HAR is applied as the acceleration bout does contain gait-related information (signals and detected steps are shown in the top panel with the HAR model output underneath, for each of the two examples). Those steps are analyzed and used to obtain values for step/stride-based biomarkers.

### Embodiments

The specific embodiments described herein are offered by way of example, not by way of limitation. Various modifications and variations of the described compositions, methods, and uses of the technology will be apparent to those skilled in the art without departing from the scope and spirit of the technology as described. Any sub-titles herein are included for convenience only, and are not to be construed as limiting the disclosure in any way.

The methods of any embodiments described herein may be provided as computer programs or as computer program products or computer readable media carrying a computer program which is arranged, when run on a computer, to perform the method(s) described above.

Unless context dictates otherwise, the descriptions and definitions of the features set out above are not limited to any particular aspect or embodiment of the invention and apply equally to all aspects and embodiments which are described.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

Other aspects and embodiments of the invention provide the aspects and embodiments described above with the term “comprising” replaced by the term “consisting of” or “consisting essentially of”, unless the context dictates otherwise.

The features disclosed in the description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.
1. In an embodiment, a computer-implemented method of processing signals from one or more wearable motion sensors to analyse gait activity of a subject is disclosed, the method comprising the steps of:
   a. receiving signals from the one or more wearable motion sensors located in any orientation on the subject, wherein the one or more wearable motion sensors comprise an accelerometer and a gyroscope; and
   b. processing the received signals, wherein processing comprises:
      i. dividing the signals from each sensor into a plurality of signal blocks comprising the signal of the respective sensor over a time interval;
      ii. determining, for at least one of the plurality of signal blocks, directional components of the signal block, wherein determining directional components comprises:
         a determining a vertical component of the signal block along the direction of gravity;
         b estimating horizontal components of the signal block in the plane perpendicular to the direction of gravity, wherein the horizontal components consist of an antero-posterior component and a medio-lateral component;
         c computing an autocorrelation of the vertical component of the signal block;
         d computing an autocorrelation of each estimated horizontal component of the signal block;
         e calculating the sum of the autocorrelations of each horizontal component obtained in step d. and of the autocorrelation of the vertical component obtained in step c.;
         f calculating the norm of the autocorrelations of each horizontal component obtained in step d. and of the autocorrelation of the vertical component obtained in step c.;
         g calculating the sum of the sum of autocorrelations obtained in step e. and of the norm of autocorrelations obtained in step f.;
         h computing an autocorrelation of the sum calculated in step g.;
         i estimating the location of the first peak of the autocorrelation obtained in step h.;
         j selecting, in each autocorrelation of the horizontal components, the peak located at the location of the first peak estimated in step i.;
         k validating the horizontal components of the signal block using one or more predetermined criteria that apply to the signs of the selected peaks of each autocorrelation of the horizontal components.
2. In an embodiment, a computer-implemented method of processing signals from one or more wearable motion sensors is disclosed, the method comprising the steps of:
   a. receiving signals from the one or more wearable motion sensors located in any orientation on the subject, wherein the one or more wearable motion sensors comprise an accelerometer and a gyroscope; and
   b. processing the received signals, wherein processing comprises:
      i. dividing the signals from each sensor into a plurality of signal blocks comprising the signal of the respective sensor over a time interval;
      ii. determining, for at least one of the plurality of signal blocks, directional components of the signal block, wherein determining directional components comprises:
         a determining a vertical component of the signal block along the direction of gravity;
         b estimating horizontal components of the signal block in the plane perpendicular to the direction of gravity, wherein the horizontal components consist of an antero-posterior component and a medio-lateral component;
         c computing an autocorrelation of the vertical component of the signal block;
         d computing an autocorrelation of each estimated horizontal component of the signal block;
         e calculating the sum of the autocorrelations of each horizontal component obtained in step d. and of the autocorrelation of the vertical component obtained in step c.;
         f calculating the norm of the autocorrelations of each horizontal component obtained in step d. and of the autocorrelation of the vertical component obtained in step c.;
         g calculating the sum of the sum of autocorrelations obtained in step e. and of the norm of autocorrelations obtained in step f.;
         h computing an autocorrelation of the sum calculated in step g.;
         i estimating the location of the first peak of the autocorrelation obtained in step h.;
         j selecting, in each autocorrelation of the horizontal components, the peak located at the location of the first peak estimated in step i.;
         k validating the horizontal components of the signal block using one or more predetermined criteria that apply to the signs of the selected peaks of each autocorrelation of the horizontal components.
3. In an embodiment, a computer-implemented method of processing signals from one or more wearable motion sensors to analyse gait activity of a subject is disclosed, the method comprising the steps of:
   a. receiving signals from the one or more wearable motion sensors located in any orientation on the subject; and
   b. processing the received signals, wherein processing comprises:
      i. dividing the signals from each sensor into a plurality of signal blocks comprising the signal of the respective sensor over a time interval;
      ii. determining, for at least one of the plurality of signal blocks, directional components of the signal block, wherein determining directional components comprises:
         a determining a vertical component of the signal block along the direction of gravity;
         b estimating horizontal components of the signal block in the plane perpendicular to the direction of gravity, wherein the horizontal components consist of an antero-posterior component and a medio-lateral component;
         c computing an autocorrelation of the vertical component of the signal block;
         d computing an autocorrelation of each estimated horizontal component of the signal block;
         e calculating the sum of the autocorrelations of each horizontal component obtained in step d. and of the autocorrelation of the vertical component obtained in step c.;
         f calculating the norm of the autocorrelations of each horizontal component obtained in step d. and of the autocorrelation of the vertical component obtained in step c.;
         g calculating the sum of the sum of autocorrelations obtained in step e. and of the norm of autocorrelations obtained in step f.;
         h computing an autocorrelation of the sum calculated in step g.;
         i estimating the location of the first peak of the autocorrelation obtained in step h.;
         j selecting, in each autocorrelation of the horizontal components, the peak located at the location of the first peak estimated in step i.;
         k validating the horizontal components of the signal block using one or more predetermined criteria that apply to the signs of the selected peaks of each autocorrelation of the horizontal components.
4. In an embodiment, a computer-implemented method of processing signals from one or more wearable motion sensors is disclosed, the method comprising the steps of:
   a. receiving signals from the one or more wearable motion sensors located in any orientation on the subject; and
   b. processing the received signals, wherein processing comprises:
      i. dividing the signals from each sensor into a plurality of signal blocks comprising the signal of the respective sensor over a time interval;
      ii. determining, for at least one of the plurality of signal blocks, directional components of the signal block, wherein determining directional components comprises:
         a determining a vertical component of the signal block along the direction of gravity;
         b estimating horizontal components of the signal block in the plane perpendicular to the direction of gravity, wherein the horizontal components consist of an antero-posterior component and a medio-lateral component;
         c computing an autocorrelation of the vertical component of the signal block;
         d computing an autocorrelation of each estimated horizontal component of the signal block;
         e calculating the sum of the autocorrelations of each horizontal component obtained in step d. and of the autocorrelation of the vertical component obtained in step c.;
         f calculating the norm of the autocorrelations of each horizontal component obtained in step d. and of the autocorrelation of the vertical component obtained in step c.;
         g calculating the sum of the sum of autocorrelations obtained in step e. and of the norm of autocorrelations obtained in step f.;
         h computing an autocorrelation of the sum calculated in step g.;
         i estimating the location of the first peak of the autocorrelation obtained in step h.;
         j selecting, in each autocorrelation of the horizontal components, the peak located at the location of the first peak estimated in step i.;
         k validating the horizontal components of the signal block using one or more predetermined criteria that apply to the signs of the selected peaks of each autocorrelation of the horizontal components.
5. In an embodiment, the method of any preceding embodiments is disclosed, wherein processing the received signals further comprises:
   a. classifying one or more of the plurality of signal blocks between a plurality of categories comprising at least a rest category and a non-rest category, the non-rest category comprising at least a straight-gait category, using the magnitude and/or standard deviation of the respective signal block;
   b. selecting one or more of a plurality of signal blocks classified as at least one of the plurality of categories;
   and wherein the step of determining directional components is performed on one or more of the selected signal blocks.
6. In an embodiment, the method of any preceding embodiments is disclosed, wherein processing the received signals further comprises:
   a. classifying one or more of the plurality of signal blocks between a plurality of categories comprising at least a rest category and a non-rest category, the non-rest category comprising at least a straight-gait category, using the magnitude and/or standard deviation of the respective signal block;
   b. selecting one or more of a plurality of signal blocks classified as at least one of the plurality of categories;
7. In an embodiment, a computer-implemented method of processing signals from one or more wearable motion sensors to analyse gait activity of a subject is disclosed, the method comprising the steps of:
   a. receiving signals from the one or more wearable motion sensors located in any orientation on the subject, wherein the one or more wearable motion sensors comprise an accelerometer and a gyroscope; and
   b. processing the received signals, wherein processing comprises:
      i. dividing the signals from each sensor into a plurality of signal blocks comprising the signal of the respective sensor over a time interval;
      ii. classifying one or more of the plurality of signal blocks between a plurality of categories comprising at least a rest category and a non-rest category, the non-rest category comprising at least a straight-gait category, using the magnitude and/or standard deviation of the respective signal block;
      iii. selecting one or more of a plurality of signal blocks classified as at least one of the plurality of categories;
      iv. determining, for at least one of the selected one or more signal blocks, directional components of the signal block, wherein determining directional components comprises:
         a determining a vertical component of the signal block along the direction of gravity;
         b estimating horizontal components of the signal block in the plane perpendicular to the direction of gravity, wherein the horizontal components consist of an antero-posterior component and a medio-lateral component;
         c computing an autocorrelation of the vertical component of the signal block;
         d computing an autocorrelation of each estimated horizontal component of the signal block;
         e calculating the sum of the autocorrelations of each horizontal component obtained in step d. and of the autocorrelation of the vertical component obtained in step c.;
         f calculating the norm of the autocorrelations of each horizontal component obtained in step d. and of the autocorrelation of the vertical component obtained in step c.;
         g calculating the sum of the sum of autocorrelations obtained in step e. and of the norm of autocorrelations obtained in step f.;
         h computing an autocorrelation of the sum calculated in step g.;
         i estimating the location of the first peak of the autocorrelation obtained in step h.;
         j selecting, in each autocorrelation of the horizontal components, the peak located at the location of the first peak estimated in step i.;
         k validating the horizontal components of the signal block using one or more predetermined criteria that apply to the signs of the selected peaks of each autocorrelation of the horizontal components.
8. In an embodiment, a computer-implemented method of processing signals from one or more wearable motion sensors is disclosed, the method comprising the steps of:
   a. receiving signals from the one or more wearable motion sensors located in any orientation on the subject, wherein the one or more wearable motion sensors comprise an accelerometer and a gyroscope; and
   b. processing the received signals, wherein processing comprises:
      i. dividing the signals from each sensor into a plurality of signal blocks comprising the signal of the respective sensor over a time interval;
      ii. classifying one or more of the plurality of signal blocks between a plurality of categories comprising at least a rest category and a non-rest category, the non-rest category comprising at least a straight-gait category, using the magnitude and/or standard deviation of the respective signal block;
      iii. selecting one or more of a plurality of signal blocks classified as at least one of the plurality of categories;
      iv. determining, for at least one of the selected one or more signal blocks, directional components of the signal block, wherein determining directional components comprises:
         a determining a vertical component of the signal block along the direction of gravity;
         b estimating horizontal components of the signal block in the plane perpendicular to the direction of gravity, wherein the horizontal components consist of an antero-posterior component and a medio-lateral component;
         c computing an autocorrelation of the vertical component of the signal block;
         d computing an autocorrelation of each estimated horizontal component of the signal block;
         e calculating the sum of the autocorrelations of each horizontal component obtained in step d. and of the autocorrelation of the vertical component obtained in step c.;
         f calculating the norm of the autocorrelations of each horizontal component obtained in step d. and of the autocorrelation of the vertical component obtained in step c.;
         g calculating the sum of the sum of autocorrelations obtained in step e. and of the norm of autocorrelations obtained in step f.;
         h computing an autocorrelation of the sum calculated in step g.;
         i estimating the location of the first peak of the autocorrelation obtained in step h.;
         j selecting, in each autocorrelation of the horizontal components, the peak located at the location of the first peak estimated in step i.;
         k validating the horizontal components of the signal block using one or more predetermined criteria that apply to the signs of the selected peaks of each autocorrelation of the horizontal components.
9. In an embodiment, a computer-implemented method of processing signals from one or more wearable motion sensors to analyse gait activity of a subject is disclosed, the method comprising the steps of:
   a. receiving signals from the one or more wearable motion sensors located in any orientation on the subject, wherein the one or more wearable motion sensors comprise an accelerometer and a gyroscope; and
   b. processing the received signals, wherein processing comprises determining directional components of the received signals, wherein determining directional components comprises:
      a determining a vertical component of the signal along the direction of gravity;
      b estimating horizontal components of the signal in the plane perpendicular to the direction of gravity, wherein the horizontal components consist of an antero-posterior component and a medio-lateral component;
      c computing an autocorrelation of the vertical component of the signal;
      d computing an autocorrelation of each estimated horizontal component of the signal;
      e calculating the sum of the autocorrelations of each horizontal component obtained in step d. and of the autocorrelation of the vertical component obtained in step c.;
      f calculating the norm of the autocorrelations of each horizontal component obtained in step d. and of the autocorrelation of the vertical component obtained in step c.;
      g calculating the sum of the sum of autocorrelations obtained in step e. and of the norm of autocorrelations obtained in step f.;
      h computing an autocorrelation of the sum calculated in step g.;
      i estimating the location of the first peak of the autocorrelation obtained in step h.;
      j selecting, in each autocorrelation of the horizontal components, the peak located at the location of the first peak estimated in step i.;
      k validating the horizontal components of the signal using one or more predetermined criteria that apply to the signs of the selected peaks of each autocorrelation of the horizontal components.
10. In an embodiment, a computer-implemented method of processing signals from one or more wearable motion sensors is disclosed, the method comprising the steps of:
   a receiving signals from the one or more wearable motion sensors located in any orientation on the subject, wherein the one or more wearable motion sensors comprise an accelerometer and a gyroscope; and
   b processing the received signals, wherein processing comprises determining directional components of the received signals, wherein determining directional components comprises:
   c determining a vertical component of the signal along the direction of gravity;
   d estimating horizontal components of the signal in the plane perpendicular to the direction of gravity, wherein the horizontal components consist of an antero-posterior component and a medio-lateral component;
   e computing an autocorrelation of the vertical component of the signal;
   f computing an autocorrelation of each estimated horizontal component of the signal;
   g calculating the sum of the autocorrelations of each horizontal component obtained in step d. and of the autocorrelation of the vertical component obtained in step c.;
   h calculating the norm of the autocorrelations of each horizontal component obtained in step d. and of the autocorrelation of the vertical component obtained in step c.;
   i calculating the sum of the sum of autocorrelations obtained in step e. and of the norm of autocorrelations obtained in step f.;
   j computing an autocorrelation of the sum calculated in step g.;
   k estimating the location of the first peak of the autocorrelation obtained in step h.;
   l selecting, in each autocorrelation of the horizontal components, the peak located at the location of the first peak estimated in step i.;
   m validating the horizontal components of the signal using one or more predetermined criteria that apply to the signs of the selected peaks of each autocorrelation of the horizontal components.
11. In an embodiment, a computer-implemented method of processing signals from one or more wearable motion sensors to analyse gait activity of a subject is disclosed, the method comprising the steps of:
   a. receiving signals from the one or more wearable motion sensors located in any orientation on the subject, wherein the one or more wearable motion sensors comprise an accelerometer and a gyroscope; and
   b. processing the received signals, wherein processing comprises:
      i. dividing the signals from at least one sensor into a plurality of signal blocks comprising the signal of the respective sensor over a time interval;
      ii. classifying one or more of the plurality of signal blocks between a plurality of categories comprising at least a rest category and a non-rest category, using the magnitude and/or standard deviation of the respective signal block;
      iii. selecting one or more of a plurality of signal blocks classified as at least one of the plurality of categories;
      iv. determining, for at least one of the selected one or more signal blocks, directional components of the signal block, wherein determining directional components comprises:
         a determining a vertical component of the signal block along the direction of gravity;
         b estimating horizontal components of the signal block in the plane perpendicular to the direction of gravity, wherein the horizontal components consist of an antero-posterior component and a medio-lateral component;
         c computing an autocorrelation of the vertical component of the signal block;
         d computing an autocorrelation of each estimated horizontal component of the signal block;
         e calculating the sum of the autocorrelations of each horizontal component obtained in step d. and of the autocorrelation of the vertical component obtained in step c.;
         f calculating the norm of the autocorrelations of each horizontal component obtained in step d. and of the autocorrelation of the vertical component obtained in step c.;
         g calculating the sum of the sum of autocorrelations obtained in step e. and of the norm of autocorrelations obtained in step f.;
         h computing an autocorrelation of the sum calculated in step g.;
         i estimating the location of the first peak of the autocorrelation obtained in step h.;
         j selecting, in each autocorrelation of the horizontal components, the peak located at the location of the first peak estimated in step i.;
         k validating the horizontal components of the signal block using one or more predetermined criteria that apply to the signs of the selected peaks of each autocorrelation of the horizontal components.
12. In an embodiment, a computer-implemented method of processing signals from one or more wearable motion sensors to analyse gait activity of a subject is disclosed, the method comprising the steps of:
   a. receiving signals from the one or more wearable motion sensors located in any orientation on the subject, wherein the one or more wearable motion sensors comprise an accelerometer and a gyroscope; and
   b. processing the received signals, wherein processing comprises:
      i. dividing the signals from each sensor into a plurality of signal blocks comprising the signal of the respective sensor over a time interval;
      ii. classifying one or more of the plurality of signal blocks between a plurality of categories comprising at least a rest category and a non-rest category, the non-rest category comprising at least a straight-gait category, using the magnitude and standard deviation of the respective signal block;
      iii. selecting one or more of a plurality of signal blocks classified as at least one of the plurality of categories;
      iv. determining, for at least one of the selected one or more signal blocks, directional components of the signal block, wherein determining directional components comprises:
         a determining a vertical component of the signal block along the direction of gravity;
         b estimating horizontal components of the signal block in the plane perpendicular to the direction of gravity, wherein the horizontal components consist of an antero-posterior component and a medio-lateral component;
         c computing an autocorrelation of the vertical component of the signal block;
         d computing an autocorrelation of each estimated horizontal component of the signal block;
         e calculating the sum of the autocorrelations of each horizontal component obtained in step d. and of the autocorrelation of the vertical component obtained in step c.;
         f calculating the norm of the autocorrelations of each horizontal component obtained in step d. and of the autocorrelation of the vertical component obtained in step c.;
         g calculating the sum of the sum of autocorrelations obtained in step e. and of the norm of autocorrelations obtained in step f.;
         h computing an autocorrelation of the sum calculated in step g.;
         i estimating the location of the first peak of the autocorrelation obtained in step h.;
         j selecting, in each autocorrelation of the horizontal components, the peak located at the location of the first peak estimated in step i.;
         k validating the horizontal components of the signal block using one or more predetermined criteria that apply to the signs of the selected peaks of each autocorrelation of the horizontal components.
13. In an embodiment, a computer-implemented method of processing signals from one or more wearable motion sensors to analyse gait activity of a subject is disclosed, the method comprising the steps of:
   a. receiving signals from the one or more wearable motion sensors located in any orientation on the subject, wherein the one or more wearable motion sensors comprise an accelerometer and a gyroscope; and
   b. processing the received signals, wherein processing comprises:
      i. dividing the signals from each sensor into a plurality of signal blocks comprising the signal of the respective sensor over a time interval;
      ii. classifying one or more of the plurality of signal blocks between a plurality of categories comprising at least a rest category and a non-rest category, the non-rest category comprising at least a straight-gait category, using the magnitude or standard deviation of the respective signal block;
      iii. selecting one or more of a plurality of signal blocks classified as at least one of the plurality of categories;
      iv. determining, for at least one of the selected one or more signal blocks, directional components of the signal block, wherein determining directional components comprises:
         a determining a vertical component of the signal block along the direction of gravity;
         b estimating horizontal components of the signal block in the plane perpendicular to the direction of gravity, wherein the horizontal components consist of an antero-posterior component and a medio-lateral component;
         c computing an autocorrelation of the vertical component of the signal block;
         d computing an autocorrelation of each estimated horizontal component of the signal block;
         e calculating the sum of the autocorrelations of each horizontal component obtained in step d. and of the autocorrelation of the vertical component obtained in step c.;
         f calculating the norm of the autocorrelations of each horizontal component obtained in step d. and of the autocorrelation of the vertical component obtained in step c.;
         g calculating the sum of the sum of autocorrelations obtained in step e. and of the norm of autocorrelations obtained in step f.;
         h computing an autocorrelation of the sum calculated in step g.;
         i estimating the location of the first peak of the autocorrelation obtained in step h.;
         j selecting, in each autocorrelation of the horizontal components, the peak located at the location of the first peak estimated in step i.;
         k validating the horizontal components of the signal block using one or more predetermined criteria that apply to the signs of the selected peaks of each autocorrelation of the horizontal components.
14. In an embodiment, the method of any preceding embodiments is disclosed, wherein the steps are performed in different order.
15. In an embodiment, the method of any preceding embodiments is disclosed, wherein the processing is performed independently using signals from an accelerometer.
16. In an embodiment, the method of any preceding embodiments is disclosed, wherein the processing is performed independently using signals from a gyroscope.
17. In an embodiment, the method of any preceding embodiments is disclosed, wherein validating the horizontal components of the signal block using one or more predetermined criteria that apply to the signs of the selected peaks of each autocorrelation of the horizontal components comprises:
   a. determining, based on the results of evaluation of the one or more predetermined criteria, that the estimated horizontal components are correctly identified, and/or
   b. determining, based on the results of evaluation of the one or more predetermined criteria, that the estimated horizontal components are incorrectly identified.
18. In an embodiment, the method of any preceding embodiments is disclosed, wherein validating the horizontal components of the signal block using one or more predetermined criteria that apply to the signs of the selected peaks of each autocorrelation of the horizontal components comprises:
   a. determining, based on the results of evaluation of the one or more predetermined criteria, that the estimated horizontal components are correctly identified, and
   b. determining, based on the results of evaluation of the one or more predetermined criteria, that the estimated horizontal components are incorrectly identified.
19. In an embodiment, the method of any preceding embodiments is disclosed, wherein validating the horizontal components of the signal block using one or more predetermined criteria that apply to the signs of the selected peaks of each autocorrelation of the horizontal components comprises:
   a. determining, based on the results of evaluation of the one or more predetermined criteria, that the estimated horizontal components are correctly identified, or
   b. determining, based on the results of evaluation of the one or more predetermined criteria, that the estimated horizontal components are incorrectly identified.
20. In an embodiment, the method of any preceding embodiments is disclosed, wherein dividing the signals from each sensor into a plurality of signal blocks comprising the signal of the respective sensor over a time interval comprises dividing the signals from each sensor into a plurality of non-overlapping signal blocks and/or a plurality of signal blocks of a predetermined duration.
21. In an embodiment, the method of any preceding embodiments is disclosed, wherein dividing the signals from each sensor into a plurality of signal blocks comprising the signal of the respective sensor over a time interval comprises dividing the signals from each sensor into a plurality of non-overlapping signal blocks and a plurality of signal blocks of a predetermined duration.
22. In an embodiment, the method of any preceding embodiments is disclosed, wherein dividing the signals from each sensor into a plurality of signal blocks comprising the signal of the respective sensor over a time interval comprises dividing the signals from each sensor into a plurality of non-overlapping signal blocks or a plurality of signal blocks of a predetermined duration.
23. In an embodiment, the method of any preceding embodiments is disclosed, wherein the rest category comprises a short-rest category and a long-rest category, based on a predetermined rest duration threshold.
24. In an embodiment, the method of any preceding embodiments is disclosed, wherein the straight-gait category comprises signal blocks with gyroscope signal magnitude below a predetermined minimum threshold.
25. In an embodiment, the method of any preceding embodiments is disclosed, further comprising, for at least one of the plurality of signal blocks, the steps of:
   a. calculating, using the determined directional components of the signal block, a step frequency;
   b. selecting one of the determined directional components of the signal block using the calculated step frequency and the computed autocorrelations of each directional component;
   c. obtaining a continuous wavelet transform of the selected directional component; d. calculating at least a first derivative of the continuous wavelet transform, and optionally a second derivative of the continuous wavelet transform;
   e. extracting, from the calculated at least first derivative and optionally second derivative of the continuous wavelet transform, one or more gait features.
26. In an embodiment, the method of any preceding embodiments is disclosed, further comprising, for at least one of the plurality of signal blocks, the steps of:
   a. calculating, using the determined directional components of the signal block, a step frequency;
   b. selecting one of the determined directional components of the signal block using the calculated step frequency and the computed autocorrelations of each directional component;
   c. obtaining a continuous wavelet transform of the selected directional component;
   d. calculating at least a first derivative of the continuous wavelet transform;
   e. extracting, from the calculated at least first derivative, one or more gait features.
27. In an embodiment, the method of any preceding embodiments is disclosed, further comprising, for each one of the plurality of signal blocks, the steps of:
   a. calculating, using the determined directional components of the signal block, a step frequency;
   b. selecting one of the determined directional components of the signal block using the calculated step frequency and the computed autocorrelations of each directional component;
   c. obtaining a continuous wavelet transform of the selected directional component;
   d. calculating at least a first derivative of the continuous wavelet transform, and optionally a second derivative of the continuous wavelet transform;
   e. extracting, from the calculated at least first derivative and optionally second derivative of the continuous wavelet transform, one or more gait features.
28. In an embodiment, the method of any of embodiments 25-27 is disclosed, wherein the one or more gait features comprise step duration, stride duration, step frequency, heel-strike events, toe-off events, stance-phase parameters (e.g. duration, average duration, frequency, average frequency, . . . ), swing-phase parameters (e.g. duration, average duration, frequency, average frequency, . . . ), indirect gait features, wherein indirect gait features can comprise the subject's cadence, fatigue, stability, rhythm/variability, asymmetry, pace, forward balance, lateral balance.
29. In an embodiment, the method of the preceding embodiment is disclosed, further comprising extracting one or more summarized gait features over the one or more signal blocks.
30. In an embodiment, the method of the preceding embodiment is disclosed, wherein the one or more summarized gait features comprise summarized metrics of the one or more gait features extracted for each of the one or more signal blocks.
31. In an embodiment, the method of the preceding embodiment is disclosed, wherein the summarized metrics comprise mean, median, trimmed versions thereof.
32. In an embodiment, the method of any of embodiments 25-31 is disclosed, wherein the step of calculating, using the determined directional components of the signal block, a step frequency comprises the step of validating the calculated step frequency, wherein the step of validating the calculated step frequency comprises the steps of:
   a. determining, for one or more neighbour signal blocks in a neighbourhood of the signal block, directional components of the neighbour signal blocks;
   b. calculating in the one or more neighbour signal blocks, using the determined directional components of the neighbour signal blocks, a neighbour step frequency;
   c. validating the calculated step frequency using the one or more calculated neighbour step frequencies, wherein the neighbourhood of a query signal block comprises bocks that are within a predetermined number of signal blocks away from the query block.
33. In an embodiment, the method of any of embodiments 25-32 is disclosed, wherein the step of calculating, using the determined directional components of the signal block, a step frequency comprises the steps of:
   a. computing a first autocorrelation of the antero-posterior component of the signal block;
   b. computing a second autocorrelation of the antero-posterior component of the signal block, wherein the second autocorrelation consists of an autocorrelation of the first autocorrelation of the antero-posterior component obtained in step a.;
   c. computing a first autocorrelation of the vertical component of the signal block;
   d. computing a second autocorrelation of the vertical component of the signal block, wherein the second autocorrelation consists of an autocorrelation of the first autocorrelation of the vertical component obtained in step c.;
   e. computing a cross-correlation of the second autocorrelation of the antero-posterior component and of the second autocorrelation of the vertical component;
   f. estimating the distance between peaks of the computed cross-correlation;
   g. calculating, using the estimated distance, the step frequency.
   In any such embodiment, the signal can be a signal received from the gyroscope and the cross-correlation of the second autocorrelation of the antero-posterior component and of the second autocorrelation of the vertical component can be a cross-correlation of the sum of the second autocorrelation of the antero-posterior component and of the second autocorrelation of the vertical component.
34. In an embodiment, the method of any embodiments 25-32 is disclosed, wherein the step of calculating, using the determined directional components of the signal block, a step frequency comprises the steps of:
   a. computing a first autocorrelation of the vertical component of the signal block;
   b. computing a second autocorrelation of the vertical component of the signal block, wherein the second autocorrelation consists of an autocorrelation of the first autocorrelation of the vertical component obtained in step a.;
   c. computing a first autocorrelation of the antero-posterior component of the signal block;
   d. computing a second autocorrelation of the antero-posterior component of the signal block, wherein the second autocorrelation consists of an autocorrelation of the first autocorrelation of the antero-posterior component obtained in step c.;
   e. computing a cross-correlation of the second autocorrelation of the antero-posterior component and of the second autocorrelation of the vertical component;
   f. estimating the distance between peaks of the computed cross-correlation;
   g. calculating, using the estimated distance, the step frequency.

In any such embodiment, the signal can be a signal received from the gyroscope and the cross-correlation of the second autocorrelation of the antero-posterior component and of the second autocorrelation of the vertical component can be a cross-correlation of the sum of the second autocorrelation of the antero-posterior component and of the second autocorrelation of the vertical component.
35. In an embodiment, the method of any embodiments 25-34 is disclosed, wherein selecting one of the determined directional components of the signal block using the calculated step frequency and the computed autocorrelations of each directional component comprises the steps of:
   a. calculating a power spectral density for each autocorrelation of each directional component;
   b. selecting an interval comprising the calculated step frequency;
   c. selecting the directional component with the highest power spectral density in the selected interval.
36. In an embodiment, the method of any embodiments 25-35 is disclosed, wherein obtaining a continuous wavelet transform of the selected directional component comprises obtaining a parameterized continuous wavelet function, and wherein obtaining the parameterized continuous wavelet function comprises estimating at least a wavelet scale and/or a wavelet sign.
37. In an embodiment, the method of any embodiments 25-35 is disclosed, wherein obtaining a continuous wavelet transform of the selected directional component comprises obtaining a parameterized continuous wavelet function.
38. In an embodiment, the method of any embodiments 25-35 is disclosed, wherein obtaining a continuous wavelet transform of the selected directional component comprises obtaining a parameterized continuous wavelet function, and wherein obtaining the parameterized continuous wavelet function comprises estimating at least a wavelet scale or a wavelet sign.
39. In an embodiment, the method of any embodiments 25-38 is disclosed, wherein the wavelet scale is a function of the calculated step frequency.
40. In an embodiment, the method of any embodiments 25-39 is disclosed, wherein estimating the wavelet sign comprises the steps of:
   a. computing a norm of the accelerometer signal of the signal block;
   b. computing a correlation of the parameterized continuous wavelet function and of the computed norm;
41. determining, based on the sign of the computed correlation, the wavelet sign. In an embodiment, the method of any embodiments 25-40 is disclosed, wherein the step of extracting, from the calculated at least first derivative and optionally second derivative of the continuous wavelet transform, one or more gait features comprises the steps of extracting gait-cycle events and validating the extracted gait-cycle events, and wherein the step of validating the extracted gait-cycle events comprise the steps of:
   a. identifying false positive and/or false negative gait-cycle events;
   b. removing false positive and/or false negative gait-cycle events; and/or
   c. validating the extracted gait-cycle events using gait-cycle events in one or more neighbour gait cycles.
42. In an embodiment, the method of any embodiments 25-40 is disclosed, wherein the step of extracting, from the calculated at least first derivative and optionally second derivative of the continuous wavelet transform, one or more gait features comprises the steps of extracting gait-cycle events and validating the extracted gait-cycle events.
43. In an embodiment, the method of any embodiments 25-40 is disclosed, wherein the step of extracting, from the calculated at least first derivative and optionally second derivative of the continuous wavelet transform, one or more gait features comprises the steps of extracting gait-cycle events and validating the extracted gait-cycle events, and wherein the step of validating the extracted gait-cycle events comprise the steps of:
   a. identifying false positive and/or false negative gait-cycle events;
   b. removing false positive and/or false negative gait-cycle events; and
   c. validating the extracted gait-cycle events using neighbour gait cycles.
43. In an embodiment, a method of diagnosing or monitoring a neurological dysfunction associated with gait activity in a subject is disclosed, the method comprising analyzing the gait activity of the subject using the method of any preceding embodiment.
44. In an embodiment, a method of diagnosing a neurological dysfunction associated with gait activity in a subject is disclosed, the method comprising analyzing the gait activity of the subject using the method of any preceding embodiment.
45. In an embodiment, a method of monitoring a neurological dysfunction associated with gait activity in a subject is disclosed, the method comprising analyzing the gait activity of the subject using the method of any preceding embodiment.
46. In an embodiment, a method of diagnosing and monitoring a neurological dysfunction associated with gait activity in a subject is disclosed, the method comprising analyzing the gait activity of the subject using the method of any preceding embodiment.
47. In an embodiment, the method of any embodiments 41-44 is disclosed, wherein the neurological dysfunction is selected from: multiple sclerosis (MS), Parkinson's disease, Huntington's disease, optionally wherein the neurological dysfunction is MS.
48. In an embodiment, the method of any embodiments 41-44 is disclosed, wherein the neurological dysfunction is selected from: multiple sclerosis (MS), Parkinson's disease, Huntington's disease.
49. In an embodiment, the method of any embodiments 41-44 is disclosed, wherein the neurological dysfunction is MS.
50. In an embodiment, a system is disclosed, the system comprising:
   a. a processor; and
   b. a computer readable medium comprising instructions that, when executed by the processor, cause the processor to perform the steps of the method of any preceding embodiments.
51. In an embodiment, a system is disclosed, the system comprising:
   a. a processor; and
   b. a computer readable medium comprising instructions that, when executed by the processor, cause the processor to perform the steps of the method of any preceding embodiments; and
   c. optionally signal acquisition means, in particular one or more wearable motion sensors.
52. In an embodiment, a computer readable medium is disclosed comprising instructions which, when executed by a computer, cause the computer to perform the steps of the method of any preceding embodiments.
53. In an embodiment, a computer program product is disclosed comprising instructions which, when the program is executed by a computer, cause the computer to perform the steps of the method of any preceding embodiments.
54. The invention as hereinbefore described.

### REFERENCES

All references cited herein are incorporated herein by reference in their entirety and for all purposes to the same extent as if each individual publication or patent or patent application was specifically and individually indicated to be incorporated by reference in its entirety.
Palmerini et al (2017), Identification of Characteristic Motor Patterns Preceding Freezing of Gait in Parkinson's Disease Using Wearable Sensors, Front. Neurol. 8:384.
Moe-Nilssen et al (2004), Estimation of gait cycle characteristics by trunk accelerometry, Journal of Biomechanics, Vol 37, Issue 1, January 2004, Pages 121-126.
McCamley et al (2012), An enhanced estimate of initial contact and final contact instants of time using lower trunk inertial sensor data, Gait Posture, Jun; 36(2):316-8.
Godfrey et al (2014), The association between retirement and age on physical activity in older adults, Age Ageing, May; 43(3):386-93.
Del Din et al (2015), Validation of an accelerometer to quantify a comprehensive battery of gait characteristics in healthy older adults and Parkinson's disease: toward clinical and at home use, IEEE J. Biomed. Health Inform. 20, 838-47.
Ning et al (2019), Appropriate mother wavelets for continuous gait event detection based on time-frequency analysis for hemiplegic and healthy individuals, Sensors (Basel), August 8; 19(16):3462.
Zhou et al (2016), Towards real-time detection of gait events on different terrains using time-frequency analysis and peak heuristics algorithm, Sensors, 16, 1634.
Pham et al (2017), Validation of a step detection algorithm during straight walking and turning in patients with Parkinson's disease and older adults using an inertial measurement unit at the lower back, Front. Neurol., Vol 8.
Caramia et al (2019), Optimizing the scale of a wavelet-based method for the detection of gait events from a waist-mounted accelerometer under different walking speeds, Sensors (Basel), April 19; 19(8): 1869.
Angelini et al (2021), A multifactorial model of Multiple Sclerosis gait and its changes across different disability levels, IEEE Trans Biomed Eng, November 21; 68(11)″:3196-3204.
Stratonovich, R. L. (1959), On the theory of optimal non-linear filtering of random functions, Theory of Probability and its Applications, 4, pp. 223-225.
Dempster, A. P. (1967), Upper and lower probabilities induced by a multivalued mapping, The Annals of Mathematical Statistics, 38(2): 325-339.
McCamley et al. (2012) An enhanced estimate of initial contact and final contact instants of time using lower trunk inertial sensor data, Gait & Posture 36 (2012) 316-318.
Cheng et al. (2017) Human Activity Recognition from Sensor-Based Large-Scale Continuous Monitoring of Parkinson's Disease Patients, 2017 IEEE/ACM International Conference on Connected Health: Applications, Systems and Engineering Technologies (CHASE).
A. Soltani et al., “Algorithms for Walking Speed Estimation Using a Lower-Back-Worn Inertial Sensor: A Cross-Validation on Speed Ranges,” in IEEE Transactions on Neural Systems and Rehabilitation Engineering, vol. 29, pp. 1955-1964, 2021, doi: 10.1109/TNSRE.2021.3111681.

What is claimed is:

1-19. (canceled)

20. A system, comprising:
at least one data processor; and
at least one memory storing instructions, which when executed by the at least one data processor, result in operations comprising:
receiving, from one or more sensors configured to be located on a subject, a signal representative of a gait activity of the subject;
extracting, from the signal, a sequence of events present in the gait activity of the subject;
determining, for an event of the sequence of events, a classification of the event as a left event or a right event, where the classification of the event is determined based at least on (i) an orientation of the one or more sensors at a time of the event and (ii) a sign of a peak of the signal at the time of the event;
determining, based at least on the classification of the event, one or more gait features of the subject; and
determining, based at least on the one or more gait features, at least one of a diagnosis, a progression, a treatment, and a treatment response for a neurological dysfunction.

21. The system of claim 20, wherein the operations further comprise:
determining, for a consecutive event preceding or succeeding the event in the sequence of events, a classification of the consecutive event as a left event or a right event; and
determining, based at least on a respective classification of the event and the consecutive event, the one or more gait features of the subject.

22. The system of claim 21, wherein the operations further comprise:
validating each of the event and the consecutive event; and
upon failing to validate the event and/or the consecutive event, excluding the event and/or the consecutive event from being used as a basis of the determining the one or more gait features of the subject.

23. The system of claim 22, wherein the operations further comprise:
determining, based at least on the classification of the event, a type of the event; and
determining, based at least on the classification of the consecutive event, a type of the consecutive event.

24. The system of claim 23, wherein the operations further comprise:
determining that the event and the consecutive event are a same type of event; and
upon determining that the event and the consecutive event are the same type of event, determining that at least one of the event and the consecutive event comprises an invalid event.

25. The system of claim 23, wherein the operations further comprise:
determining that an ordering of the event and the consecutive event fails to conform to a predetermined order of the type of event and the type of the consecutive event; and
upon determining that the ordering of the event and the consecutive event fails to conform to the predetermined order, determining that at least one of the event and the consecutive event comprise an invalid event.

26. The system of claim 25, wherein the ordering of the event and the consecutive event fails to conform to the predetermined order due to an absence of one or more intervening types of events present in the predetermined order.

27. The system of claim 20, wherein the determining the classification of the event includes
determining that the orientation of the one or more sensors at the time of the event is such that a vertical direction is negative; and
in response to determining that the orientation of the one or more sensors at the time of the event is such that the vertical direction is negative,
determining that the event is a left event upon determining that the sign of peak of the signal at the time of the event is positive; and
determining that the event is a right event upon determining that the sign the peak of the signal at the time of the event is negative.

28. The system of claim 20, wherein the determining the classification of the event includes
determining that the orientation of the one or more sensors at the time of the event is such that a vertical direction is positive; and
in response to determining that the orientation of the one or more sensors at the time of the event is such that the vertical direction is positive,
determining that the event is a right event upon determining that the sign of peak of the signal at the time of the event is positive; and
determining that the event is a left event upon determining that the sign the peak of the signal at the time of the event is negative.

29. The system of claim 20, wherein the event comprises a heel-strike event or a toe-off event.

30. The system of claim 20, wherein the one or more gait features include at least one of cadence, fatigue, stability, rhythm, variability, asymmetry, pace, forward balance, and lateral balance.

31. The system of claim 20, wherein the operations further comprise:
dividing the signal into a plurality of signal blocks;
classifying each signal block of the plurality of signal blocks as a rest block or a non-rest block;
validating one or more of the plurality of signal blocks classified as a non-rest block; and
for each validated non-rest block, classifying the validated non-rest block as a straight-gait block or a non-straight gait block; and
determining, based at least on one or more validated non-rest blocks classified as a straight-gait block, one or more gait features of the subject.

32. The system of claim 31, wherein a human activity recognition (HAR) model is applied to validate the one or more of the plurality of signal blocks classified as a non-rest block.

33. The system of claim 31, wherein the classifying each signal block as a rest block or a non-rest block includes
classifying, based at least on a magnitude of an accelerometer signal and/or a gyroscope signal in a signal block, the signal block as a rest block or a non-rest block.

34. The system of claim 31, wherein the classifying each validated non-rest block as a straight-gait block or a non-straight gait block includes
classifying the validated non-rest block as a straight-gait block based at least on a presence of motion of a threshold magnitude in a single direction of movement, and
classifying the validated non-rest block as a non-straight gait block based at least on a presence of motion in multiple directions of movement.

35. The system of claim 31, wherein the operations further comprise:
determining, based at least on the plurality of signal blocks, an environment in which the subject performs the gait activity; and
determining, based at least on the environment of the subject, at least one of the diagnosis, the progression, the treatment, and the treatment response for the neurological dysfunction.

36. The system of claim 35, wherein the environment comprises a supervised environment, an unsupervised environment, and/or a passive monitoring environment.

37. The system of claim 35, wherein the environment includes one or more features comprising at least one of a quantity of turns, a duration of turns, a quantity of walking bouts, and/or a duration of turns.

38. A computer-implemented method, comprising:
receiving, from one or more sensors configured to be located on a subject, a signal representative of a gait activity of the subject;
extracting, from the signal, a sequence of events present in the gait activity of the subject;
determining, for an event of the sequence of events, a classification of the event as a left event or a right event, where the classification of the event is determined based at least on (i) an orientation of the one or more sensors at a time of the event and (ii) a sign of a peak of the signal at the time of the event;
determining, based at least on the classification of the event, one or more gait features of the subject; and
determining, based at least on the one or more gait features, at least one of a diagnosis, a progression, a treatment, and a treatment response for a neurological dysfunction.

39. A non-transitory computer readable medium storing instructions, which when executed by at least one data processor, result in operations comprising:
receiving, from one or more sensors configured to be located on a subject, a signal representative of a gait activity of the subject;
extracting, from the signal, a sequence of events present in the gait activity of the subject;
determining, for an event of the sequence of events, a classification of the event as a left event or a right event, where the classification of the event is determined based at least on (i) an orientation of the one or more sensors at a time of the event and (ii) a sign of a peak of the signal at the time of the event;
determining, based at least on the classification of the event, one or more gait features of the subject; and
determining, based at least on the one or more gait features, at least one of a diagnosis, a progression, a treatment, and a treatment response for a neurological dysfunction.
